SEQUENCE LISTING (2 sequences)

SEQ ID NO: 1 - DNA, 1918 bp, Homo sapiens
  CDS (31)...(1056)
tctctgctggtgttcatcagaacagacaccatggcagagcatgattaccatgaagactatgggttcagcagtttcaatgacagcagccaggaggagcatcaagacttcctgcagttcagcaaggtctttctgccctgcatgtacctggtggtgtttgtctgtggtctggtggggaactctctggtgctggtcatatccatcttctaccataagttgcagagcctgacggatgtgttcctggtgaacctacccctggctgacctggtgtttgtctgcactctgcccttctgggcctatgcaggcatccatgaatgggtgtttggccaggtcatgtgcaagagcctactgggcatctacactattaacttctacacgtccatgctcatcctcacctgcatcactgtggatcgtttcattgtagtggttaaggccaccaaggcctacaaccagcaagccaagaggatgacctggggcaaggtcaccagcttgctcatctgggtgatatccctgctggtttccttgccccaaattatctatggcaatgtctttaatctcgacaagctcatatgtggttaccatgacgaggcaatttccactgtggttcttgccacccagatgacactggggttcttcttgccactgctcaccatgattgtctgctattcagtcataatcaaaacactgcttcatgctggaggcttccagaagcacagatctctaaagatcatcttcctggtgatggctgtgttcctgctgacccagatgcccttcaacctcatgaagttcatccgcagcacacactgggaatactatgccatgaccagctttcactacaccatcatggtgacagaggccatcgcatacctgagggcctgccttaaccctgtgctctatgcctttgtcagcctgaagtttcgaaagaacttctggaaacttgtgaaggacattggttgcctcccttaccttggggtctcacatcaatggaaatcttctgaggacaattccaagactttttctgcctcccacaatgtggaggccaccagcatgttccagttataggccttgccagggtttcgagaagctgctctggaatttgcaagtcatggctgtgccctcttgatgtggtgaggcaggctttgtttatagcttgcgcattctcatggagaagttatcagacactctggctggtttggaatgcttcttctcaggcatgaacatgtactgttctcttcttgaacactcatgctgaaagcccaagtagggggtctaaaatttttaaggactttccttcctccatctccaagaatgctgaaaccaagggggatgacatgtgactcctatgatctcaggttctccttgattgggactggggctgaaggttgaagaggtgagcacggccaacaaagctgttgatggtaggtggcacactgggtgcccaagctcagaaggctcttctgactactgggcaaagagtgtagatcagagcagcagtgaaaacaagtgctggcaccaccaggcacctcacagaaatgagatcaggctctgcctcaccttggggcttgacttttgtataggtagatgttcagattgctttgattaatccagaataactagcaccagggactatgaatgggcaaaactgaattataagaggctgataattccagtggtccatggaatgcttgaaaaatgtgcaaaacagcgtttaagactgtaatgaatctaagcagcatttctgaagtggactctttggtggctttgcattttaaaaatgaaattttccaatgtctgccacacaaacgtatgtaaatgtatatacccacacacatacacacatatgtcatatattactagcatatgagtttcatagctaagaaataaaactgttaaagtctccaaaaaaaaaaaaaaaaaaaaa

SEQ ID NO: 2 - protein, 342 aa, Homo sapiens
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

(19) United States
(12) Patent Application Publication
(10) Pub. No.: US 2003/0203450 A1
(43) Pub. Date: Oct. 30, 2003

(54) STRL33, a human fusion accessory factor associated with HIV infection

(76) Inventors: Joshua M. Farber, Washington, DC (US); Fang Liao, Taipei (TW); Ghalib Alkhatib, Carmel, IN (US); Edward A. Berger, Rockville, MD (US)

Correspondence Address: NATIONAL INSTITUTE OF HEALTH, C/O NEEDLE & ROSENBERG, P.C., SUITE 1000, 999 PEACHTREE STREET, ATLANTA, GA, 30303, US

(21) Appl. No.: 10/458,110

(22) Filed: Jun. 9, 2003

Related U.S. Application Data
(63) Continuation of application No. 09/402,275, filed on Dec. 22, 1999.
Application No. 09/402,275 is a 371 of international application No. PCT/US98/06517, filed on Mar. 31, 1998.
(60) Provisional application No. 60/042,880, filed on Mar. 31, 1997.

(51) Int. Cl. A01K 67/027; C07H 21/04; C12P 21/02; C12N 5/06; C07K 14/705

(52) U.S. Cl. 435/69.1; 435/320.1; 435/354; 530/350; 536/23.5; 800/10; 800/14; 800/18

(57) ABSTRACT

The susceptibility to human immunodeficiency virus (HIV) infection depends on the cell surface expression of the human CD4 molecule and a human fusion accessory factor associated with HIV infection (STRL33). STRL33 is a member of the 7-transmembrane segment superfamily of G-protein-coupled cell surface molecules. STRL33 plays a role in the membrane fusion step of HIV infection for both TCL-tropic and M-tropic variants of HIV. The invention provides STRL33 polypeptide and polynucleotide sequences encoding STRL33 polypeptide. The establishment of stable, nonhuman cell lines and transgenic mammals having cells that coexpress human CD4 and STRL33 provides valuable tools for the continuing research of HIV infection and the development of more effective anti-HIV therapeutics. In addition, antibodies against STRL33, isolated and purified peptide fragments of STRL33, and STRL33-binding biologic agents, capable of blocking membrane fusion between HIV and target cells represent potential anti-HIV therapeutics.

### 1. FIELD OF THE INVENTION

The present invention pertains generally to in vitro and in vivo models for the study of human immunodeficiency virus (HIV) infection and the effectiveness of anti-HIV therapeutics and specifically to a polypeptide, designated STRL33, which plays a role in the membrane fusion step of HIV infection.

### 2. BACKGROUND OF THE INVENTION

The HIV infection cycle begins with the entry of the virus into the target cell. The human CD4 molecule is the primary receptor recognized by HIV. The binding of the HIV envelope glycoprotein (env) to the CD4 receptor results in the fusion of virus and cell membranes, which in turn facilitates virus entry into the host. The eventual expression of env on the surface of the HIV-infected host cell enables this cell to fuse with uninfected, CD4-positive cells, thereby spreading the virus.

Recent studies have shown that this HIV fusion process occurs with a wide range of human cell types that either express human CD4 endogenously or have been engineered to express human CD4. The fusion process, however, does not occur with nonhuman cell types engineered to express human CD4. Although such nonhuman cells can still bind env, membrane fusion does not follow. The disparity between human and nonhuman cell types exists apparently because membrane fusion requires the coexpression of human CD4 and an accessory factor specific to human cell types. Because they lack this accessory factor, nonhuman cell types engineered to express only human CD4 are incapable of membrane fusion, and are thus nonpermissive for HIV infection. To date there has been no report of any stable, nonhuman cell line that is permissive for HIV infection as a result of human CD4 and STRL33 coexpression.

The importance of human CD4 and STRL33 coexpression also impacts the establishment of a successful small animal model. The development of a small animal model is crucial to the study of HIV infection and the effectiveness of anti-HIV therapeutics. In recent years, researchers have bred transgenic animals having cells that express human CD4. See, for example, Dunn et al., Human immunodeficiency virus type I infection of human CD4-transgenic rabbits, J. Gen. Vir. 76:1327-1336 (1995); Snyder et al., Development and Tissue-Specific Expression of Human CD4 in Transgenic Rabbits, Mol. Reprod. & Devel. 40:419-428 (1995); Killeen et al., Regulated Expression of human CD4 Rescues Helper T-Cell Development in Mice Lacking Expression of Endogenous CD4, EMBO J. 12:1547-1553 (1993); Forte et al., Human CD4 Produced in Lymphoid Cells of Transgenic Mice Binds HIV p120 and Modifies the Subsets of Mouse T-Cell Populations, Immunogenetics 38:455-459 (1993). These animals, however, have low susceptibility to HIV infection, presumably because of the lack expression of a co-receptor for. To date, there has been no report of any transgenic animal that is significantly susceptible to HIV infection as a result of human CD4 and STRL33 coexpression.

Without an effective vaccine, the number of individuals infected with HIV will likely increase substantially. Furthermore, in the absence of effective therapy, most individuals infected with HIV will develop acquired immune deficiency syndrome (AIDS) and succumb to either opportunistic infections and malignancies that result from the deterioration of the immune system, or the direct pathogenic effects of the virus. Despite the present availability of some anti-HIV agents that slow disease progression, a pressing need remains for more effective therapeutics and drug combinations.

It is apparent from the foregoing that a need exists for in vitro and in vivo models suitable to the study of HIV infection and the effectiveness of anti-HIV therapeutics. By the same token, the need remains for more effective anti-HIV therapeutics.

### SUMMARY OF THE INVENTION

The susceptibility to HIV infection depends on the cell surface expression of the human CD4 molecule and a heretofore unidentified human fusion accessory factor. The present invention provides a novel fusion accessory factor, designated STRL33. Comparison of the nucleotide sequence of the cDNA encoding STRL33 against a computer database revealed that STRL33 is a member of the 7-transmembrane segment superfamily of G-protein-coupled cell surface molecules (GPCR). Many of the superfamily members function as ligand receptors in relation, for example, to peptide hormones, neurotransmitters, and chemokines. STRL33 has no known ligand.

The identification of STRL33 adds to the recent discoveries on the roles of chemokine receptors in the pathobiology of HIV-1 infection. STRL33 is a novel GPCR that can function with CD4 to mediate fusion with cells bearing HIV-1 Envs from both laboratory-adapted TCL-tropic variants and from M-tropic variants. In this regard, STRL33 can mediate fusion with a wider range of Envs than can the major cofactors fusin/CXCR4 and CCR5. While the role of STRL33 in the biology of viral infection is unknown, the present invention demonstrates both that STRL33 is functional as a cofactor for HIV-1 Env-mediated fusion and that the STRL33 gene is expressed in cells and tissues that are natural targets for HIV-1.

A key aspect of the present invention is the discovery that STRL33 plays a role in the membrane fusion step of HIV infection. The establishment of stable, nonhuman cell lines and transgenic mammals having cells that coexpress human CD4 and STRL33 provides valuable tools for the continuing research of HIV infection and the development of more effective anti-HIV therapeutics. In addition, antibodies which bind STRL33, isolated and purified peptide fragments of STRL33, and STRL33-binding agents, capable of blocking membrane fusion between HIV and target cells represent potential anti-HIV therapeutics. The invention provides STRL33 polynucleotides and polypeptides as well.

In one embodiment, the invention provides nonhuman cell lines, the cells of which contain DNA encoding STRL33 and express both human CD4 and STRL33. In another embodiment, the invention provides transgenic non-human animals having cells that coexpress human CD4 and STRL33.

A further objective of the present invention is to provide antibodies, preferably monoclonal antibodies, that bind STRL33 and that block membrane fusion between HIV and a target cell or between an HIV infected cell and an uninfected CD4 positive cell.

Yet another objective is the isolation and purification of peptide fragments of STRL33 that block membrane fusion between HIV and a target cell. Also included are fragments of HIV env polypeptide that block membrane fusion between HIV and target cell or between an HIV infected cell and an uninfected CD4 positive cell.

It also is an objective of the present invention to isolate and purify STRL33-binding agents, both biologic and chemical compounds, that block membrane fusion between HIV and a target cell or between an HIV infected cell and an uninfected CD4 positive cell.

In accomplishing these and other objectives, there is provided a stable, nonhuman cell line, the cells of which contain DNA encoding a human accessory fusion factor associated with HIV infection (STRL33), and coexpress human CD4 and STRL33; a transgenic non-human mammal comprised of cells that coexpress human CD4 and STRL33; an antibody against STRL33 that blocks membrane fusion between HIV and a target cell; a monoclonal antibody against STRL33 that blocks membrane fusion between HIV and a target cell; an isolated and purified peptide fragment of STRL33, wherein said peptide fragment blocks membrane fusion between HIV and a target cell; and an isolated and purified STRL33-binding biologic agent, wherein said biologic agent blocks membrane fusion between HIV and a target cell.

Also included in the invention are methods of treating a subject having or at risk of having an HIV-related disorder associated with expression of STRL33 comprising administering to an HIV infected or susceptible cell of the subject, a reagent that suppresses STRL33. Therapeutic methods of the invention using an anti-STRL33 antibody are described. Further, the invention also includes methods of gene therapy wherein an antisense nucleic acid that hybridizes to a STRL33 nucleic acid is administered to a subject. The reagent is introduced into the cell using a carrier, such as a vector. Administration of the reagent can be in vivo or ex vivo.

In another embodiment, the invention provides a method for detecting susceptibility of a cell to HIV infection by detecting fusion of a test cell with a cell that expresses HIV-env. Also included are methods of identifying compositions which either bind to STRL33 or block membrane fusion between HIV and a target cell or between an HIV-infected cell and a STRL33 positive uninfected cell. Preferably the STRL33 cell is also CD4 positive.

In yet another embodiment the invention provides a method for modulating a T-cell response by administration of STRL33 agonists or antagnoists.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows the alignment of the STRL33 predicted amino acid sequence with the selected GPCRs STRL22, GPR-9-6, EB11, IL8RB, CXCR4, CCR3, CCR5 and IL8RA. Numbers at the right indicate the positions of the residues at the end of each line of sequence. Solid backgrounds highlight matches between STRL33 and the other receptors. Dots indicate gaps introduced for optimal alignment. Putative TMDs I-VII are indicated by bars. The alignments were generated using the PileUp program of the Wisconsin Sequence Analysis Package of Genetics Computer Group, Madison, Wis.

FIG. 2 shows the expression STRL33 and other GPCR genes. FIG. 2A. The expression of STRL-33, genes for known chemokine receptors, and genes for selected orphan GPCRs in leukocytes. Fifteen micrograms of total RNA were electrophoresed on 1.2% agarose-fornaldehyde gels, transferred to nitrocellulose membranes and hybridized with the probes indicated on the left. A total of six membranes were used for hybridizations, and adequate removal of signal was documented before repeat probings. Film exposure times ranged from overnight for the IL8RA and IL8RB blots to 13 d for the CXCR4 blot. Probings were done using an oligonucleotide complementary to 18S RNA in order to demonstrate amounts of RNA loaded per lane and a representative blot is shown. FIG. 2B. The expression of STRL33 in human tissues. Blots were prepared by the supplier (Clontech) from 1.2% agarose-formaldehyde gels containing approximately 2 μg poly (A)⁺ RNA per lane. Hybridizations were done using a ³²P-labelled STRL33 ORF probe and blots were washed according to the supplier's instructions. Membranes were exposed to film using an intensifying screen. The blot prepared from lymphoid tissue (left) was exposed for 2 d, and the blot from other selected tissues (right) was exposed for 8 d.

FIG. 3 shows the activity of STRL33 as a fusion cofactor. FIG. 3A. NIH 3T3 cells were transfected with DNAs encoding fusin/CXCR4 or CCR5 or STRL33, and infected with vaccinia recombinants encoding CD4 and T7 RNA polymerase. HeLa cells were infected with a vaccinia recombinant containing LacZ under control of a T7 promoter and infected separately with vaccinia recombinants encoding the indicated Envs. Unc is a mutant Env that cannot be cleaved to gp120 and gp41 and cannot mediate fusion. Cell fusion was quantified by measuring β-Gal activity. NIH 3T3 cells transfected with the STRL33 cDNA but not infected with virus vCB-3 encoding CD4 did not fuse with cells expressing any of the Envs. Results of one experiment are shown. STRL33 also mediated fusion with cells expressing both TCL-tropic and M-tropic Envs in four additional experiments. FIG. 3B. Jurkat cell line JC0.1, transfected with vector control, and Jurkat cell line JC3.9, transfected with the STRL33.1 cDNA, were infected with vaccinia recombinants encoding the T7 RNA polymerase and CD4. Jurkat cells were incubated with recombinant vaccinia-infected HeLa cells and fusion was measured as in A.

FIG. 4 shows the nucleotide and deduced amino acids sequence of STRL33.

### DETAILED DESCRIPTION OF PREFERRED EMBODIMENTS

In accordance with the present invention, the STRL33 refers to a cellular protein of the 7-transmembrane segment superfamily of G-protein-coupled cell surface molecules that is associated with the fusion of virus and target cell membranes in HIV infection. The essential role of STRL33 in the membrane fusion step of HIV infection was determined by functional assay of the effects of recombinant STRL33 (i.e., assay by vaccinia cell fusion system or HIV infection).

### cDNA ENCODING STRL33 AND STRL33 POLYPEPTIDE

To identify novel chemokine receptors expressed in T cells, we used RT-PCR with poly(A)+RNA prepared from tumor infiltrating lymphocytes (TIL line F9) and pools of degenerate primers based on conserved sequences in the transmembrane domains (TMDs) of known chemokine receptors. A sequence encoding a novel GPCR, designated STRL33, for seven transmembrane domain receptor from lymphocytes, clone 33, is isolated. Southern blot analysis of human genomic DNA digested with BamH I, Hind III and Pst I revealed a single STRL33 gene, and using STRL33-specific primers and DNA prepared from a panel of human-hamster hybrid cell lines, STRL33 was localized to chromosome 3. This raises the possibility that STRL33 is in the cluster of genes for chemokine receptors CCR1, 2, 3, and 5 at 3p21, although the CCR proteins show >50% amino acid identity among themselves, demonstrating significantly closer relationships than what is seen in comparisons with STRL33 (see below).

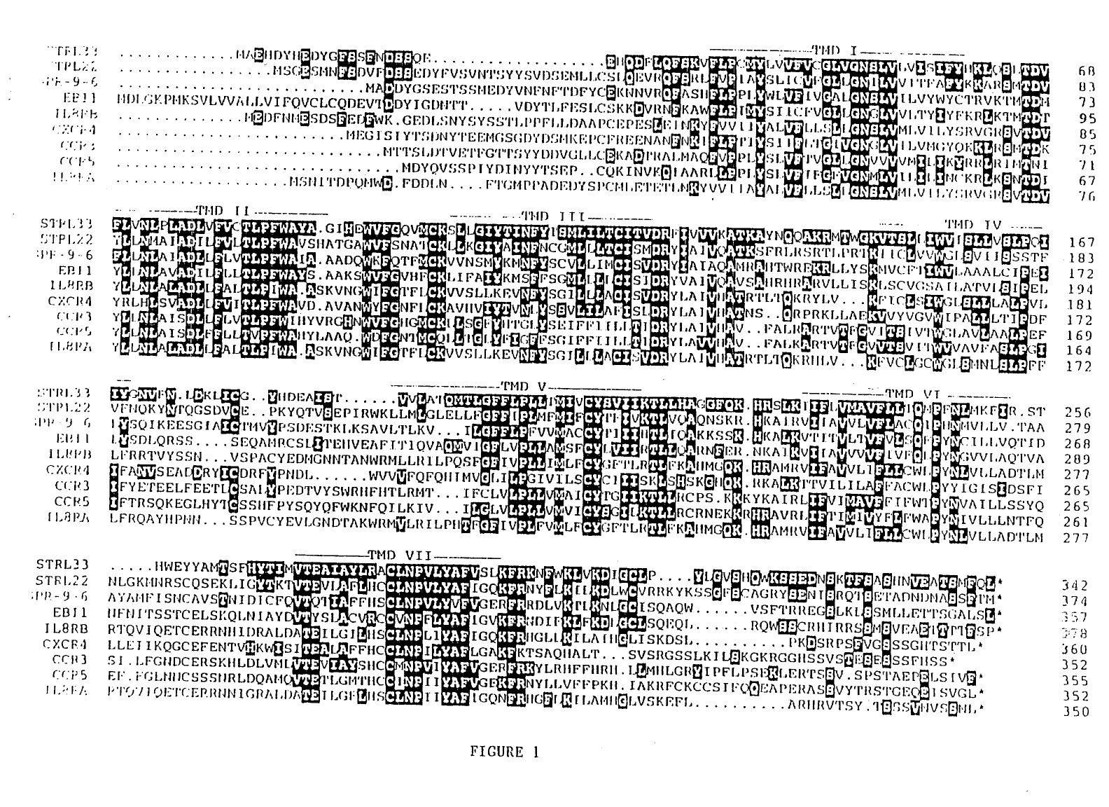

Screening of a non-arnplified lambda cDNA library prepared from F9 TIL revealed an abundance for STRL33 mRNA of approximately 0.01%. Ten cDNA clones were isolated and by restriction enzyme digestion, 3 size classes were identified. Representative cDNAs from each class, STRL33.1, 33.2, and 33.3, respectively, were evaluated in more detail by restriction analysis and partial sequencing, revealing that size differences among the clones were due to 5′ non-translated regions that differed not only in length but in their sequences. The complete sequence of STRL33.1, the sequence of the 5′ non-translated region of STRL33.2, and the sequence of the 5′ non-translated region and open reading frame (ORF) of 33.3 have been submitted to GenBank with accession numbers U73529, U73530, and U73531 respectively. STRL33.1 contained 1897 nucleotides, excluding the poly(A) tail, with an ORF encoding a predicted protein of 342 amino acids (FIG. 1). The predicted initiator codon was in a favorable context for initiation (33) and could be assigned unambiguously since, with the reading frame fixed by comparison with other GPCRs, it was the first ATG following an in-frame stop codon in cDNA 33.3, and the first in-frame ATG in cDNAs 33.1 and 33.2. ORF sequence was also determined from cDNAs other than 33.1, and the sequence of the entire 33.1 ORF was confirmed in independent clones. Differences were noted, however, between the ORFs of 33.1 and 33.3 at two positions: a second position change in the 25th codon (GAC→GCC), replacing D25 with A, and a silent third position change in the 103 rd codon. The silent change in the 103 rd codon was present in a second independent clone, while the 25th codon change has not been independently verified. At least the silent change probably represents a true polymorphism in the F9 TIL. The 5′ non-translated regions of 33.1, 0.2, and 0.3 were 30, 135, and 1462 nucleotides respectively, and sequence comparisons revealed that differences among these regions were due, at least in part, to alternative splicing. Not surprisingly, the long additional 5′ non-translated sequence of 33.3 contained many ATG sequence (but no significant ORFs) suggesting that the 33.3 mRNA may not be translated efficiently, and although we could detect a 33.3-specific mRNA by Northern analysis (see below), the 33.3 cDNA may be derived from an incompletely processed mRNA. Extensive processing, yielding mRNAs with alternative 5′ exons, is well documented among the chemoattractant receptors (2).

Comparison of STRL33 with sequences in the databases using BLAST (34) revealed no identical sequences but greatest similarity to orphan GPCRs and related chemokine receptors, and alignments between STRL33 and selected related sequences is shown in FIG. 1. Percent identities between STRL33 and orphan receptors STRL22 (22), GPR-9-6 (unpublished, GenBank accession number U45982) and EBI1 (35) are 37%, 32% and 32% respectively and between STRL33 and chemokine receptors IL8RB (CXCR2), fusin/CXCR4, CCR3, CCR5 and IL8RA (CXCR1) are 30%, 30%, 30%, 29% and 28% respectively. STRL22 is an orphan receptor that, like STRL33, was isolated from the F9 TIL (22).

FIG. 1 shows that similarities among the receptors are greatest in the TMDs, as is typical of GPCRs. Like other GPCRs, STRL33 includes a site for N-linked glycosylation in the N terminal domain (N16), cysteines in extracellular loops one and two (C102 and C180), and multiple serines in the carboxy terminal domain (1). While there is no signature sequence motif for the chemokine receptors, the STRL33 sequence does contain some features characteristic of chemokine receptors, including an acidic N-terminal domain with paired acidic residues (E8 and D9, E21 and E22) (2), a short basic third intracellular loop, an alanine in place of the proline that is conserved in non-chemokine receptor GPCRs in the second intracellular loop (A134), a paired cysteine and tyrosine in TMD V (C210 and Y211) and a cysteine in TMD VII (C282). In contrast, some residues typical for chemokine receptors are absent from STRL33, including cysteine residues in the N-terminal domain and in the third extracellular loop. Multiple sequence alignment (PileUp, Genetics Computer Croup, Madison, Wis.) places STRL33 in a group of orphan receptors including STRL22, GPR-9-6 and EB11 separate from the groupings of the CXCRs on one hand and the CCRs on the other.

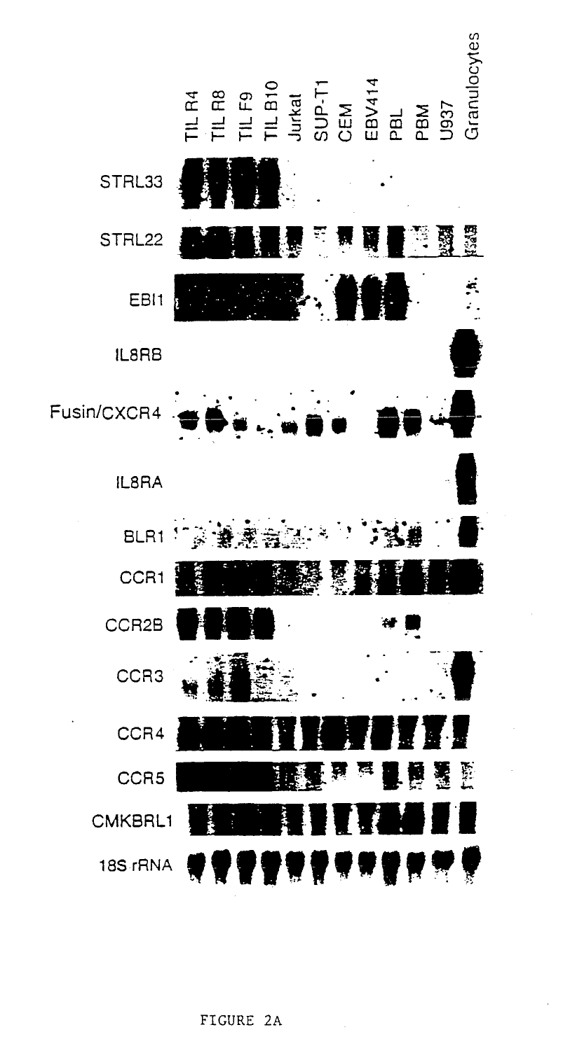

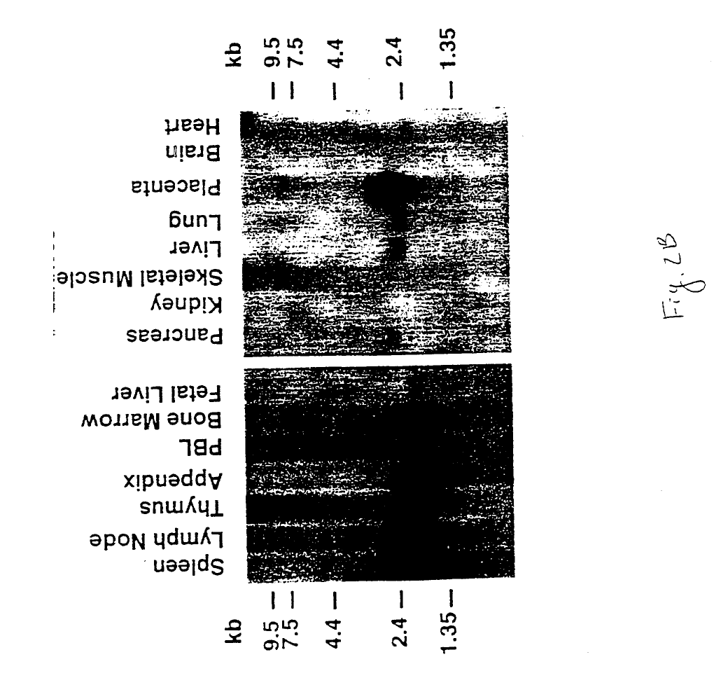

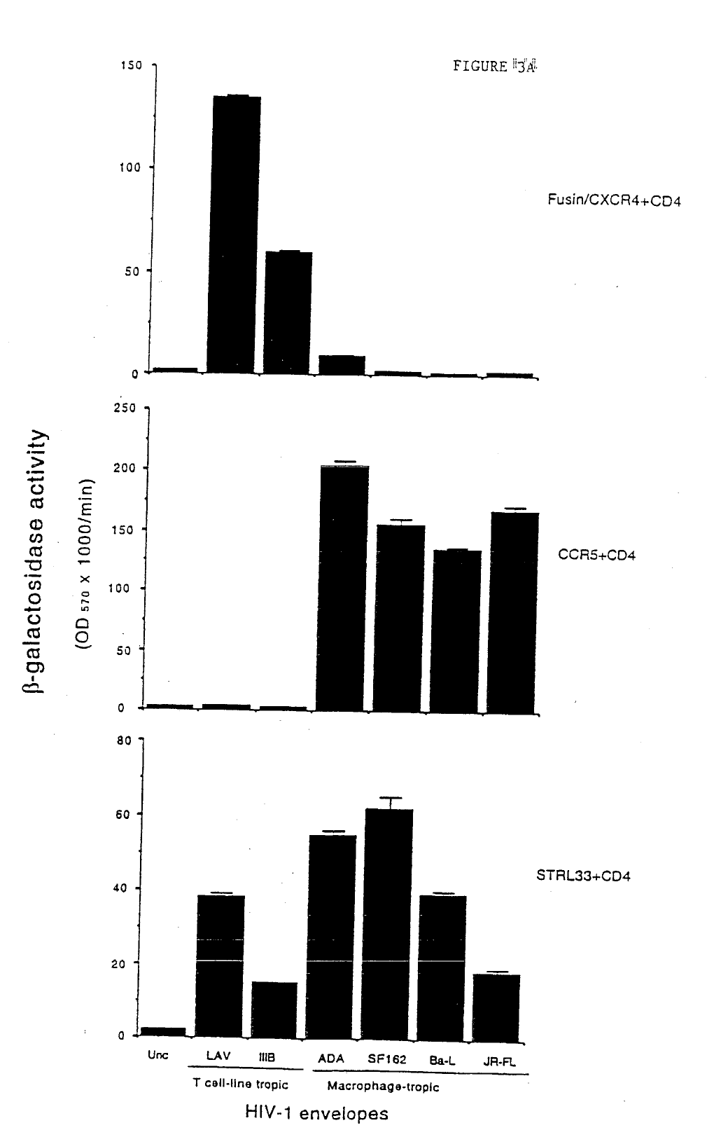

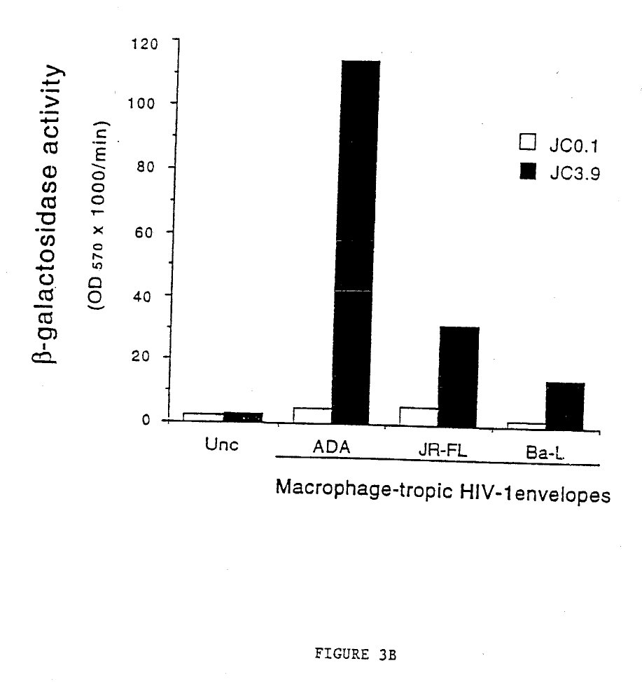

RNA expression was analyzed by Northern blot of total RNA for STRL33 and other related receptors in leukocyte populations and lines and in human tissues. As shown in FIG. 2A, the STRL33 cDNA probe hybridized to a broad band at approximately 2 kb that was prominent in both CD4+ (R4, F9 and B10) and CD8+ (R8) TIL with low level signal in PBL but not in other cells tested, including immortalized CD4+ T cell lines. Using a probe from the 5′ non-translated sequences specific for the STRL33.3 mRNA, we also detected a band at approximately 3.6 kb in the F9 and B10 TIL after long exposure, not shown in FIG. 2A.

FIG. 2A shows significant differences in expression of various chemokine receptor and orphan receptor genes among the leukocytes. Of particular interest is the demonstration of heterogeneity of receptor gene expression among T cell preparations. In general, receptor gene expression is higher among the TIL than in T cell lines or PBL, although even among the TIL there are significant differences. The CD4+ F9 TIL, for example, show a significant signal for CCR3. This is noteworthy, since although CCR3 can serve in vitro as a coreceptor for HIV-1 (13), speculation on a role for CCR3 in HIV-1 infection has been constrained by the assumption that CCR3 expression is limited to eosinophils.

FIG. 2B shows the expression of the STRL33 gene in selected human tissues. There is an mRNA species of approximately 2.1 kb prominently expressed in lymphoid tissue; a prominent species of approximately 2.5 kb in placenta; low-abundance species of 2.1-2.4 kb expressed in pancreas, liver, lung and heart; and low-abundance, larger species in a variety of tissues. The conspicuous expression of the STRL33 gene in T cells and lymphoid tissues is consistent with the presumption that STRL33 is a chemokine receptor.

HEK 293 cells and Jurkat cells were transfected with expression vectors containing the STRL33.1 ORF and vector control DNA, and cell lines were derived as described in the Examples. Cell lines expressing the highest levels of STRL33 RNA were tested in a fluorometeric calcium flux assay for responses to chemokines. The STRL33-transfected HEK 293 and/or Jurkat cell lines were tested with platelet factor 4, IL8, IP-10, HuMig, SDF-1, MIP-1∝, MIP-1β, RANTES, MCP-1, MCP-2, MCP-3, MCP4, I309 and lymphotactin and no responses were found.

The present invention provides substantially pure STRL33 polypeptide. The term “substantially pure” as used herein refers to STRL33 which is substantially free of other proteins, lipids, carbohydrates or other materials with which it is naturally associated. The substantially pure polypeptide will yield a single major band on a reducing or a non-reducing polyacrylamide gel. The purity of the STRL33 polypeptide can also be determined by amino-terminal amino acid sequence analysis. STRL33 polypeptide includes functional fragments of the polypeptide, as long as the activity of STRL33 remains. Smaller peptides containing the biological activity of STRL33 are included in the invention.

The invention provides polynucleotides encoding the STRL33 polypeptide. These polynucleotides include DNA, cDNA and RNA sequences which encode STRL33. It is understood that all polynucleotides encoding all or a portion of STRL33 are also included herein, as long as they encode a polypeptide with STRL33 activity. Such polynucleotides include naturally occurring, synthetic, and intentionally manipulated polynucleotides. For example, STRL33 polynucleotide may be subjected to site-directed mutagenesis. The polynucleotide sequence for STRL33 also includes antisense sequences. The polynucleotides of the invention include sequences that are degenerate as a result of the genetic code. There are 20 natural amino acids, most of which are specified by more than one codon. Therefore, all degenerate nucleotide sequences are included in the invention as long as the amino acid sequence of STRL33 polypeptide encoded by the nucleotide sequence is functionally unchanged.

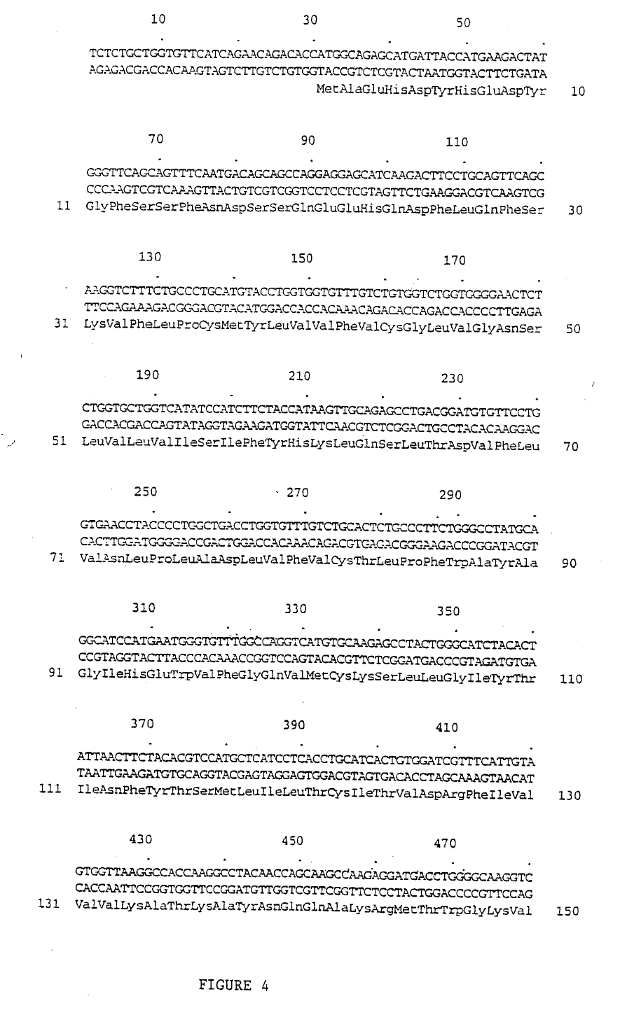

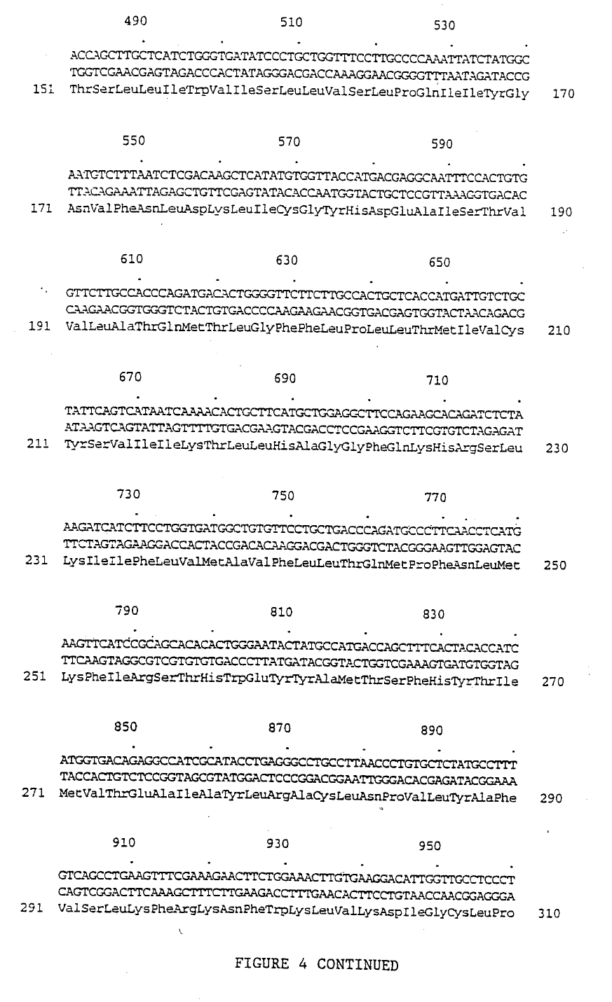

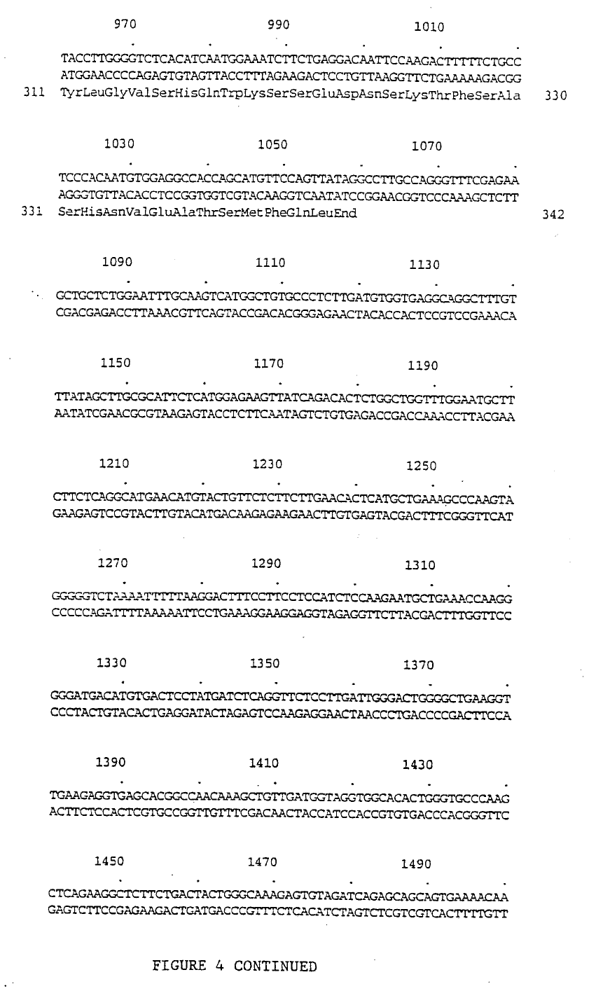

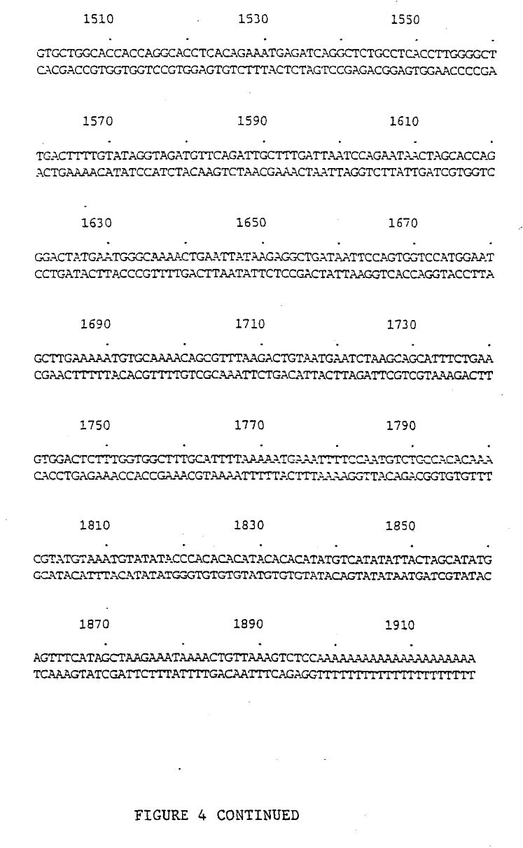

The polynucleotide encoding STRL33 includes SEQ ID NO:1 (FIG. 4) as well as nucleic acid sequences complementary to FIG. 4. A complementary sequence may include an antisense nucleotide. When the sequence is RNA, the deoxynucleotides A, G, C, and T of the nucleic acid of FIG. 4 are replaced by ribonucleotides A, G, C, and U, respectively. Also included in the invention are fragments of the above-described nucleic acid sequences that are at least 15 bases in length, which is sufficient to permit the fragment to selectively hybridize to DNA that encodes the protein of FIG. 4 under physiological conditions. Specifically, the fragments should hybridize to DNA encoding STRL33 protein under moderately stringent conditions.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Minor modifications of the STRL33 primary amino acid sequence may result in proteins which have substantially equivalent activity as compared to the STRL33 polypeptide described herein. Such proteins include those as defined by the term having essentially the amino acid sequence of the polypeptide of FIG. 4. Such modifications may be deliberate, as by site-directed mutagenesis, or may be spontaneous. All of the polypeptides produced by these modifications are included herein as long as the biological activity of STRL33 still exists. Further, deletion of one or more amino acids can also result in a modification of the structure of the resultant molecule without significantly altering its biological activity. This can lead to the development of a smaller active molecule which would have broader utility. For example, one can remove amino or carboxy terminal amino acids which are not required for STRL33 biological activity.

The STRL33 polypeptide of the invention encoded by the polynucleotide of the invention includes the disclosed sequence (FIG. 4) and conservative variations thereof. The term “conservative variation” as used herein denotes the replacement of an amino acid residue by another, biologically similar residue. Examples of conservative variations include the substitution of one hydrophobic residue such as isoleucine, valine, leucine or methionine for another, or the substitution of one polar residue for another, such as the substitution of arginine for lysine, glutamic for aspartic acid, or glutamine for asparagine, and the like. The term “conservative variation” also includes the use of a substituted amino acid in place of an unsubstituted parent amino acid provided that antibodies raised to the substituted polypeptide also immunoreact with the unsubstituted polypeptide.

DNA sequences of the invention can be obtained by several methods. For example, the DNA can be isolated using hybridization techniques which are well known in the art. These include, but are not limited to: 1) hybridization of genomic or cDNA libraries with probes to detect homologous nucleotide sequences, 2) polymerase chain reaction (PCR) on genomic DNA or cDNA using primers capable of annealing to the DNA sequence of interest, and 3) antibody screening of expression libraries to detect cloned DNA fragments with shared structural features.

Preferably the STRL33 polynucleotide of the invention is derived from a mammalian organism, and most preferably from human. Screening procedures which rely on nucleic acid hybridization make it possible to isolate any gene sequence from any organism, provided the appropriate probe is available. Oligonucleotide probes, which correspond to a part of the sequence encoding the protein in question, can be synthesized chemically. This requires that short, oligopeptide stretches of amino acid sequence must be known. The DNA sequence encoding the protein can be deduced from the genetic code, however, the degeneracy of the code must be taken into account. It is possible to perform a mixed addition reaction when the sequence is degenerate. This includes a heterogeneous mixture of denatured double-stranded DNA. For such screening, hybridization is preferably performed on either single-stranded DNA or denatured double-stranded DNA.

Hybridization is particularly useful in the detection of cDNA clones derived from sources where an extremely low amount of mRNA sequences relating to the polypeptide of interest are present. In other words, by using stringent hybridization conditions directed to avoid non-specific binding, it is possible, for example, to allow the autoradiographic visualization of a specific cDNA clone by the hybridization of the target DNA to that single probe in the mixture which is its complete complement (Wallace, et al., Nucl. Acid Res., 9:879, 1981; Maniatis, et al., Molecular Cloning: A Laboratory Manual, Cold Spring Harbor, N.Y. 1989).

The development of specific DNA sequences encoding STRL33 can also be obtained by: 1) isolation of double-stranded DNA sequences from the genomic DNA; 2) chemical manufacture of a DNA sequence to provide the necessary codons for the polypeptide of interest; and 3) in vitro synthesis of a double-stranded DNA sequence by reverse transcription of mRNA isolated from a eukaryotic donor cell. In the latter case, a double-stranded DNA complement of mRNA is eventually formed which is generally referred to as cDNA.

Of the three above-noted methods for developing specific DNA sequences for use in recombinant procedures, the isolation of genomic DNA isolates is the least common. This is especially true when it is desirable to obtain the microbial expression of mammalian polypeptides due to the presence of introns.

The synthesis of DNA sequences is frequently the method of choice when the entire sequence of amino acid residues of the desired polypeptide product is known. When the entire sequence of amino acid residues of the desired polypeptide is not known, the direct synthesis of DNA sequences is not possible and the method of choice is the synthesis of cDNA sequences. Among the standard procedures for isolating cDNA sequences of interest is the formation of plasmid- or phage-carrying cDNA libraries which are derived from reverse transcription of mRNA which is abundant in donor cells that have a high level of genetic expression. When used in combination with polymerase chain reaction technology, even rare expression products can be cloned. In those cases where significant portions of the amino acid sequence of the polypeptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single-stranded form (Jay, et al., Nucl. Acid Res., 11:2325, 1983).

A cDNA expression library, such as lambda gt11, can be screened indirectly for STRL33 peptides having at least one epitope, using antibodies specific for STRL33. Such antibodies can be either polyclonally or monoclonally derived and used to detect expression product indicative of the presence of STRL33 cDNA.

DNA sequences encoding STRL33 can be expressed in vitro by DNA transfer into a suitable host cell. “Host cells” are cells in which a vector can be propagated and its DNA expressed. The term also includes any progeny of the subject host cell. It is understood that all progeny may not be identical to the parental cell since there may be mutations that occur during replication. However, such progeny are included when the term “host cell” is used. Methods of stable transfer, meaning that the foreign DNA is continuously maintained in the host, are known in the art.

In the present invention, the STRL33 polynucleotide sequences may be inserted into a recombinant expression vector. The term “recombinant expression vector” refers to a plasmid, virus or other vehicle known in the art that has been manipulated by insertion or incorporation of the STRL33 genetic sequences. Such expression vectors contain a promoter sequence which facilitates the efficient transcription of the inserted genetic sequence of the host. The expression vector typically contains an origin of replication, a promoter, as well as specific genes which allow phenotypic selection of the transformed cells. Vectors suitable for use in the present invention include, but are not limited to the T7-based expression vector for expression in bacteria (Rosenberg, et al., Gene, 56:125, 1987), the pMSXND expression vector for expression in mammalian cells (Lee and Nathans, J. Biol. Chem., 263:3521, 1988) and baculovirus-derived vectors for expression in insect cells. The DNA segment can be present in the vector operably linked to regulatory elements, for example, a promoter (e.g., T7, metallothionein I, or polyhedrin promoters).

Polynucleotide sequences encoding STRL33 can be expressed in either prokaryotes or eukaryotes. Hosts can include microbial, yeast, insect and mammalian organisms. Methods of expressing DNA sequences having eukaryotic or viral sequences in prokaryotes are well known in the art. Biologically functional viral and plasmid DNA vectors capable of expression and replication in a host are known in the art. Such vectors are used to incorporate DNA sequences of the invention.

Transformation of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransformed with DNA sequences encoding the STRL33 of the invention, and a second foreign DNA molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the protein. (see for example, Eukaryotic Viral Vectors, Cold Spring Harbor Laboratory, Gluzman ed., 1982).

Isolation and purification of microbial expressed polypeptide, or fragments thereof, provided by the invention, may be carried out by conventional means including preparative chromatography and immunological separations involving monoclonal or polyclonal antibodies.

### STRL33 FUNCTIONAL ASSAY

In one embodiment the invention provides a method for detecting susceptibility of a cell to HIV infection. The method includes incubating a first cell to be tested for susceptibility, with a second cell which is known to express HIV-env, under suitable conditions to allow fusion of the two cells (see below for an example of suitable conditions). Susceptibility is indicated by detecting fusion of the cells. Detection is preferably by a reporter gene, as described below for lacZ, however, other reporter means are known in the art and are discussed in the present specification under “Screen For STRL33 Blocking Agents”.

The demonstrations that receptors for both CXC and CC chemokines can function as cofactors for HIV-1 entry into cells led to testing STRL33 in an assay designed to detect fusion between two cell populations: NIH 3T3 cells expressing T7 RNA polymerase, human CD4, and either STRL33 or fusin/CXCR4 or CCR5 , and HeLa cells expressing Envs from HIV-1 isolates with distinct tropisms (see references in 32) and containing the Lac Z gene under control of a T7 promoter. DNAs encoding the GPCRs were introduced into NIH 3T3 cells by transfection and the DNAs encoding other proteins were introduced into cells using recombinant vaccinia viruses. Fusion between the transfected/infected NIH 3T3 and the Env-expressing cells resulted in expression of β-Gal. Envs examined included the prototypic TCL-tropic LAV and IIIB and the prototypic M-tropic ADA, SF162, Ba-L, and JR-FL. Recent data using the ADA Env show that it differs somewhat from the other M-tropic Envs in demonstrating low-level fusion with T cell lines (32), and with cells expressing fusin/CXCR4 (8 and see below). As a negative control, we used the Unc Env, a mutant protein that cannot mediate fusion due to a deletion of the gp120/gp41 cleavage site. Murine NIH/3T3 cells or human HeLa cells are coinfected with various vaccinia viruses: vTF7-3 (containing the T7 RNA polymerase gene); vCB3 (containing the human CD4 gene); vSTRL33 (containing the STRL33 gene); and vaccinia WR (a negative control). A different cell population is coinfected with various vaccinia viruses: vCB-21R (containing the E. coli lacZ gene under the transcriptional control of a T7 promoter (P_{T7}-lacZ) along with either vSC60 (containing the HIV-1 env gene (IIIB isolate)) or vCB-16 (a negative control, containing a mutant env gene encoding an uncleavable, nonfusogenic unc/env). The cell populations are incubated overnight at 31° C. to allow expression of the vaccinia-encoded proteins. The cells are washed and mixtures are prepared in 96-well microtiter plates. Each well contains equal numbers of the indicated pairs of T7 RNA polymerase-containing cells and lacZ gene-containing cells. Replicate plates are incubated for 4 hours at 37° C. to allow fusion. Samples on one plate are treated with NP40 and aliquots are assayed for β-galactosidase activity using a 96-well absorbance reader. Samples on the second plate are stained with crystal violet for syncytia analysis by light microscopy.

The results of the fusion assays are shown in FIG. 3A. NIH 3T3 cells expressing CD4 plus fusin/CXCR4 fused well with cells expressing Envs from LAV and IIIB and much less well with cells expressing the ADA Env; β-Gal activity with the JR-FL and Ba-L Envs was not above the background seen with the non-fusogenic Unc Env. Cells expressing CD4 plus CCR5 fused well with cells expressing the Envs from the M-tropic variants ADA, SF162, Ba-L and JR-FL and not the Envs from LAV and IIIB. These results are consistent with previous reports (8, 11-15).

In contrast to the restricted specificities of fusin/CXCR4 and CCR5, STRL33 functioned with CD4 as a fusion cofactor for cells expressing Envs from both TCL-tropic and M-tropic variants. Negligible β-Gal activity, equivalent to levels seen using the Unc Env, was detected in fusion assays using CD4-expressing NIH 3T3 cells transfected with a control vector lacking the STRL33 cDNA insert, or in assays using NIH 3T3 cells transfected with the STRL33 cDNA but not expressing CD4.

STRL33 was also examined in stable lines of Jurkat cells that had been transfected with DNA encoding STRL33 or with a vector control and cloned by limiting dilution and hygromycin selection. The Jurkat cell lines were infected with recombinant vaccinia viruses encoding T7 RNA polymerase and CD4, and then mixed with cells that had been infected with the recombinant vaccinia virus encoding β-Gal and infected separately with recombinant viruses encoding Envs Unc, ADA, JR-FL or Ba-L. As shown in FIG. 3B, the STRL33-transfected Jurkat cells could be fused with cells expressing the Envs from the M-tropic strains ADA, JR-FL and Ba-L but not with cells expressing Unc. The vector control-transfected Jurkat cells did not support fusion with these Envs. When the TCL-tropic LAV Env was examined, comparable levels of fusion were observed for the STRL33-transfected and the vector control-transfected cells, presumably because Jurkat cells express fusin/CXCR4 (see FIG. 2A).

Preferably, in the fusion method of the invention, the first or the second cell contains a reporter means and at least the test cell, or first cell, is a T cell. A first or second cell typically includes a T-cell for in vivo use and NIH-3T3 cells or any of the cells described in the following section for use in vitro. The fusion method described herein is also particularly useful for screening fusion inhibiting agents and pharmacological agents useful in treatment of HIV infection, both prophylactically and after infection. Examples of these agents are described in more detail below, and include but are not limited to peptides, antibodies, peptidomimetics, and chemical compounds.

Cell Lines

In one embodiment, the present invention provides human and nonhuman cell lines, the cells of which contain DNA encoding STRL33 and coexpress human CD4 and STRL33. The cells which provide the starting material in which STRL33 are expressed must be STRL33 negative, but can be either CD4 positive or CD4 negative cells. Suitable cell types include but are not limited to, cells of the following types: NIH-3T3 murine fibroblasts, quail QT6 quail cells, canine Cf2Th thymocytes, MV1 Lu mink lung cells, Sf9 insect cells, primary T-cells, and human T-cell lines such as H9, U-87 MG glioma cell, and CEM. Such cells are described, for example, in the Cell Line Catalog of the American Type Culture Collection (ATCC, Rockville, Md., USA, 20852). The stable transfer of genes into mammalian cells has been well described in the art. See, for example, Ausubel et al., Introduction of DNA Into Mammalian Cells, in CURRENT PROTOCOLS IN MOLECULAR BIOLOGY, sections 9.5.1-9.5.6 (John Wiley & Sons, Inc. 1995).

STRL33 can be expressed using inducible or constituitive regulatory elements for such expression. Commonly used constituitive or inducible promoters, for example, are known in the art. The desired protein encoding sequence and an operably linked promoter may be introduced into a recipient cell either as a non-replicating DNA (or RNA) molecule, which may either be a linear molecule or, more preferably, a closed covalent circular molecule. Since such molecules are incapable of autonomous replication, the expression of the desired molecule may occur through the transient expression of the introduced sequence. Alternatively, permanent expression may occur through the integration of the introduced sequence into the host chromosome. Therefore the cells can be transformed stably or transiently.

An example of a vector that may be employed is one which is capable of integrating the desired gene sequences into the host cell chromosome. Cells which have stably integrated the introduced DNA into their chromosomes can be selected by also introducing one or more markers which allow for selection of host cells which contain the expression vector.

The marker may complement an auxotrophy in the host (such as leu2, or ura3, which are common yeast auxotrophic markers), biocide resistance, e.g., antibiotics, or heavy metals, such as copper, or the like. The selectable marker gene can either be directly linked to the DNA gene sequences to be expressed, or introduced into the same cell by co-transfection.

In a preferred embodiment, the introduced sequence will be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

For a mammalian host, several possible vector systems are available for expression. One class of vectors utilize DNA elements which provide autonomously replicating extra-chromosomal plasmids, derived from animal viruses such as bovine papilloma virus, polyoma virus, adenovirus, or SV40 virus. A second class of vectors include vaccinia virus expression vectors. A third class of vectors relies upon the integration of the desired gene sequences into the host chromosome. Cells which have stably integrated the introduced DNA into their chromosomes may be selected by also introducing one or more markers (e.g., an exogenous gene) which allow selection of host cells which contain the expression vector. The marker may provide for prototropy to an auxotrophic host, biocide resistance, e.g., antibiotics, or heavy metals, such as copper or the like. The selectable marker gene can either be directly linked to the DNA sequences to be expressed, or introduced into the same cell by co-transformation. Additional elements may also be needed for optimal synthesis of mRNA. These elements may include splice signals, as well as transcription promoters, enhancers, and termination signals. The cDNA expression vectors incorporating such elements include those described by Okayama, H., Mol. Cell. Biol., 3:280 (1983), and others. Once the vector or DNA sequence containing the construct has been prepared for expression, the DNA construct may be introduced (transformed) into an appropriate host. Various techniques may be employed, such as protoplast fusion, calcium phosphate precipitation, electroporation or other conventional techniques.

### TRANSGENIC ANIMALS

In another embodiment, the present invention relates to transgenic non-human animals having cells that coexpress human CD4 and STRL33. Such transgenic animals represent a model system for the study of HIV infection and the development of more effective anti-HIV therapeutics. The transgenic animals of the invention can be produced from animals which express CD4 or from animals that do not express CD4. However, while the invention provides transgenic animals that express STRL33 alone, the preferred invention transgenic non-human animal co-expresses CD4 and STRL33. The invention also envisions transgenic animals that express other co-factors necessary for HIV-env-mediated cell fusion.

The term “animal” here denotes all mammalian species except human. It also includes an individual animal in all stages of development, including embryonic and fetal stages. Farm animals (pigs, goats, sheep, cows, horses, rabbits and the like), rodents (such as mice), and domestic pets (for example, cats and dogs) are included within the scope of the present invention.

A “transgenic” animal is any animal containing cells that bear genetic information received, directly or indirectly, by deliberate genetic manipulation at the subcellular level, such as by microinjection or infection with recombinant virus. “Transgenic” in the present context does not encompass classical crossbreeding or in vitro fertilization, but rather denotes animals in which one or more cells receive a recombinant DNA molecule. Although it is highly preferred that this molecule be integrated within the animal's chromosomes, the present invention also contemplates the use of extrachromosomally replicating DNA sequences, such as might be engineered into yeast artificial chromosomes.

The term “transgenic animal” also includes a “germ cell line” transgenic animal. A germ cell line transgenic animal is a transgenic animal in which the genetic information has been taken up and incorporated into a germ line cell, therefore conferring the ability to transfer the information to offspring. If such offspring in fact possess some or all of that information, then they, too, are transgenic animals.

It is highly preferred that the transgenic animals of the present invention be produced by introducing into single cell embryos DNA encoding STRL33 and DNA encoding human CD4, in a manner such that these polynucleotides are stably integrated into the DNA of germ line cells of the mature animal and inherited in normal mendelian fashion. Advances in technologies for embryo micromanipulation now permit introduction of heterologous DNA into fertilized mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals. Those techniques as well known. For instance, reviews of standard laboratory procedures for microinjection of heterologous DNAs into mammalian (mouse, pig, rabbit, sheep, goat, cow) fertilized ova include: Hogan et al., MANIPULATING THE MOUSE EMBRYO (Cold Spring Harbor Press 1986); Krimpenfort et al., Bio/Technology 9:86 (1991); Palmiter et al., Cell 41:343 (1985); Kraemer et al., GENETIC MANIPULATION OF THE EARLY MAMMALIAN EMBRYO (Cold Spring Harbor Laboratory Press 1985); Hammer et al., Nature, 315:680 (1985); Purcel et al., Science, 244:1281 (1986); Wagner et al., U.S. Pat. No.5,175,385; Krimpenfort et al., U.S. Pat. No. 5,175,384, the respective contents of which are incorporated by reference. The cDNA encoding STRL33 can be fused in proper reading frame under the transcriptional and translational control of a vector to produce a genetic construct that is then amplified, for example, by preparation in a bacterial vector, according to conventional methods. See, for example, the standard work: Sambrook et al., MOLECULAR CLONING: A LABORATORY MANUAL (Cold Spring Harbor Press 1989), the contents of which are incorporated by reference. The amplified construct is thereafter excised from the vector and purified for use in producing transgenic animals.

Production of transgenic animals containing the gene for human CD4 have been described. See Snyder et al., supra; Dunn et al., supra, the contents of which therefore are incorporated by reference.

The term “transgenic” as used herein additionally includes any organism whose genome has been altered by in vitro manipulation of the early embryo or fertilized egg or by any transgenic technology to induce a specific gene knockout. The term “gene knockout” as used herein, refers to the targeted disruption of a gene in vivo with complete loss of function that has been achieved by any transgenic technology familiar to those in the art. In one embodiment, transgenic animals having gene knockouts are those in which the target gene has been rendered nonfunctional by an insertion targeted to the gene to be rendered non-functional by homologous recombination. As used herein, the term “transgenic” includes any transgenic technology familiar to those in the art which can produce an organism carrying an introduced transgene or one in which an endogenous gene has been rendered non-functional or Aknocked out.

The transgene to be used in the practice of the subject invention is a DNA sequence comprising a modified STRL33 coding sequence. In a preferred embodiment, the STRL33 gene is disrupted by homologous targeting in embryonic stem cells. For example, the entire mature C-terminal region of the STRL33 gene may be deleted as described in the examples below. Optionally, the STRL33 disruption or deletion may be accompanied by insertion of or replacement with other DNA sequences, such as a non-functional STRL33 sequence. In other embodiments, the transgene comprises DNA antisense to the coding sequence for STRL33. In another embodiment, the transgene comprises DNA encoding an antibody or receptor peptide sequence which is able to bind to STRL33. Where appropriate, DNA sequences that encode proteins having STRL33 activity but differ in nucleic acid sequence due to the degeneracy of the genetic code may also be used herein, as may truncated forms, allelic variants and interspecies homologues.

### ANTIBODIES AGAINST STRL33 INHIBIT FUSION

In another embodiment, the present invention provides to antibodies against STRL33 that block env-mediated membrane fusion (I) associated with HIV entry into a human CD4-positive target cell or (ii) between an HIV-infected cell and an uninfected human CD4-positive target cell. Such antibodies are useful as research and diagnostic tools in the study of HIV infection and the development of more effective anti-HIV therapeutics. In addition, pharmaceutical compositions comprising antibodies against STRL33 may represent effective anti-HIV therapeutics.

A target cell typically includes a T-cell for in vivo use and NIH-3T3 cells or any of the above-listed cells for use in vitro. Antibodies of the invention include polyclonal antibodies, monoclonal antibodies, and fragments of polyclonal and monoclonal antibodies.

The STRL33 polypeptides of the invention can also be used to produce antibodies which are immunoreactive or bind to epitopes of the STRL33 polypeptides. Antibody which consists essentially of pooled monoclonal antibodies with different epitopic specificities, as well as distinct monoclonal antibody preparations are provided. Monoclonal antibodies are made from antigen containing fragments of the protein by methods well known in the art (Kohler, et al., Nature, 256:495, 1975; Current Protocols in Molecular Biology, Ausubel, et al., ed., 1989).

The term “antibody” as used in this invention includes intact molecules as well as fragments thereof, such as Fab, F(ab′)₂, and Fv which are capable of binding the epitopic determinant. These antibody fragments retain some ability to selectively bind with its antigen or receptor and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York (1988), incorporated herein by reference).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibodies which bind to the STRL33 polypeptide of the invention can be prepared using an intact polypeptide or fragments containing small peptides of interest as the immunizing antigen. The polypeptide or a peptide used to immunize an animal can be derived from translated cDNA or chemical synthesis which can be conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

If desired, polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1994, incorporated by reference).

It is also possible to use the anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

The preparation of polyclonal antibodies is well-known to those skilled in the art. See, for example, Green et al., Production of polyclonal Antisera, in IMMUNOCHEMICAL PROTOCOLS (Manson, ed.), pages 1-5 (Humana Press 1992); Coligan et al., Production of Polyclonal Antisera in Rabbits, Rats, Mice and Hamsters, in CURRENT PROTOCOLS IN IMMUNOLOGY, section 2.4.1 (1992), which are hereby incorporated by reference.

The preparation of monoclonal antibodies likewise is conventional. See, for example, Kohler & Milstein, Nature 256:495 (1975); Coligan et al., sections 2.5.1-2.6.7; and Harlow et al., ANTIBODIES: A LABORATORY MANUAL, page 726 (Cold Spring Harbor Pub. 1988), which are hereby incorporated by reference. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., Purification of Immunoglobulin G (IgG), in METHODS IN MOLECULAR BIOLOGY, VOL. 10, pages 79-104 (Humana Press 1992). Methods of in vitro and in vivo multiplication of monoclonal antibodies is well-known to those skilled in the art. Multiplication in vitro may be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Therapeutic applications are conceivable for the antibodies of the present invention. For example, antibodies of the present invention may also be derived from subhuman primate antibody. General techniques for raising therapeutically useful antibodies in baboons may be found, for example, in Goldenberg et al., International Patent Publication WO 91/11465 (1991) and Losman et al., Int. J. Cancer 46:310 (1990), which are hereby incorporated by reference.

Alternatively, a therapeutically useful anti-STRL33 antibody may be derived from a “humanized” monoclonal antibody. Humanized monoclonal antibodies are produced by transferring mouse complementary determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989), which is hereby incorporated in its entirety by reference. Techniques for producing humanized monoclonal antibodies are described, for example, by Jones et al., Nature 321: 522 (1986); Riechmann et al., Nature 332: 323 (1988); Verhoeyen et al., Science 239: 1534 (1988); Carter et al., Proc. Nat'l Acad. Sci. USA 89: 4285 (1992); Sandhu, Crit. Rev. Biotech. 12: 437 (1992); and Singer et al., J. Immunol. 150: 2844 (1993), which are hereby incorporated by reference.

Antibodies of the invention also may be derived from human antibody fragments isolated from a combinatorial immunoglobulin library. See, for example, Barbas et al., METHODS: A COMPANION TO METHODS IN ENZYMOLOGY, VOL.2, page 119 (1991); Winter et al.,Ann. Rev. Immunol. 12: 433 (1994), which are hereby incorporated by reference. Cloning and expression vectors that are useful for producing a human immunoglobulin phage library can be obtained, for example, from STRATAGENE Cloning Systems (La Jolla, Calif.).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994); Lonberg et al., Nature 368:856 (1994); and Taylor et al., Int. Immunol. 6:579 (1994), which are hereby incorporated by reference.

Antibody fragments of the present invention can be prepared by proteolytic hydrolysis of the antibody or by expression in E. Coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using papain produces two monovalent Fab fragments and an Fc fragment directly. These methods are described, for example, by Goldenberg, U.S. Pat. Nos. 4,036,945 and 4,331,647, and references contained therein. These patents are hereby incorporated in their entireties by reference. See also Nisonhoff et al., Arch. Biochem. Biophys. 89:230 (1960); Porter, Biochem. J. 73:119 (1959); Edelman et al., METHODS IN ENZYMOLOGY, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, e.g., Sandhu, supra. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow et al., METHODS: A COMPANION TO METHODS IN ENZYMOLOGY, VOL. 2, page 97 (1991); Bird et al., Science 242:423-426 (1988); Ladner et al., U.S. Pat. No. 4,946,778; Pack et al., Bio/Technology 11: 1271-77 (1993); and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., METHODS: A COMPANION TO METHODS IN ENZYMOLOGY, VOL. 2, page 106 (1991).

It is also envisioned that antibodies included in the invention may block HIV-env mediated cell fusion or infection by blocking the interaction between CD4, STRL33 and HIV, without actually “binding” to STRL33. Therefore, all of the above descriptions regarding antibodies that bind to STRL33 also apply to antibodies that block HIV-env mediated infection or fusion.

### PEPTIDE FRAGMENTS OF STRL33

In another embodiment, the present invention relates to substantially purified peptide fragments of STRL33 that block membrane fusion between HIV and a target cell or cell fusion between an HIV-infected cell and a susceptible uninfected cell. A “susceptible” uninfected cell should express both CD4 and STRL33. Such peptide fragments could represent research and diagnostic tools in the study of HIV infection and the development of more effective anti-HIV therapeutics. In addition, pharmaceutical compositions comprising isolated and purified peptide fragments of STRL33 may represent effective anti-HIV therapeutics.

It is also envisioned that a peptide fragment useful for blocking membrane fusion as described herein, includes fragments of HIV env.

The term “substantially purified” as used herein refers to a molecule, such as a peptide that is substantially free of other proteins, lipids, carbohydrates, nucleic acids, and other biological materials with which it is naturally associated. For example, a substantially pure molecule, such as a polypeptide, can be at least 60%, by dry weight, the molecule of interest. One skilled in the art can purify STRL33 peptides using standard protein purification methods and the purity of the polypeptides can be determined using standard methods including, e.g., polyacrylamide gel electrophoresis (e.g., SDS-PAGE), column chromatography (e.g., high performance liquid chromatography (HPLC)), and amino-terminal amino acid sequence analysis.

The invention relates not only to fragments of naturally-occurring STRL33, but also to STRL33 mutants and chemically synthesized derivatives of STRL33 that block membrane fusion between HIV and a target cell.

For example, changes in the amino acid sequence of STRL33 are contemplated in the present invention. STRL33 can be altered by changing the DNA encoding the protein. Preferably, only conservative amino acid alterations are undertaken, using amino acids that have the same or similar properties. Illustrative amino acid substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine.

Additionally, other variants and fragments of STRL33 can be used in the present invention. Variants include analogs, homologs, derivatives, muteins and mimetics of STRL33 that retain the ability to block membrane fusion. Fragments of the STRL33 refer to portions of the amino acid sequence of STRL33 that also retain this ability. The variants and fragments can be generated directly from STRL33 itself by chemical modification, by proteolytic enzyme digestion, or by combinations thereof. Additionally, genetic engineering techniques, as well as methods of synthesizing polypeptides directly from amino acid residues, can be employed.

Non-peptide compounds that mimic the binding and function of STRL33 (“mimetics”) can be produced by the approach outlined in Saragovi et al., Science 253: 792-95 (1991). Mimetics are molecules which mimic elements of protein secondary structure. See, for example, Johnson et al.,“Peptide Turn Mimetics,” in BIOTECHNOLOGY AND PHARMACY, Pezzuto et al., Eds., (Chapman and Hall, New York 1993). The underlying rationale behind the use of peptide mimetics is that the peptide backbone of proteins exists chiefly to orient amino acid side chains in such a way as to facilitate molecular interactions. For the purposes of the present invention, appropriate mimetics can be considered to be the equivalent of STRL33 itself.

Variants and fragments also can be created by recombinant techniques employing genomic or cDNA cloning methods. Site-specific and region-directed mutagenesis techniques can be employed. See CURRENT PROTOCOLS IN MOLECULAR BIOLOGY vol. 1, ch. 8 (Ausubel et al. eds., J. Wiley & Sons 1989 & Supp. 1990-93); PROTEIN ENGINEERING (Oxender & Fox eds., A. Liss, Inc. 1987). In addition, linker-scanning and PCR-mediated techniques can be employed for mutagenesis. See PCR TECHNOLOGY (Erlich ed., Stockton Press 1989); CURRENT PROTOCOLS IN MOLECULAR BIOLOGY, vols. 1 & 2, supra. Protein sequencing, structure and modeling approaches for use with any of the above techniques are disclosed in PROTEIN ENGINEERING, loc. cit., and CURRENT PROTOCOLS IN MOLECULAR BIOLOGY, vols. 1 & 2, supra.

If the compounds described above are employed, the skilled artisan can routinely insure that such compounds are amenable for use with the present invention utilizing cell fusion assays known in the art, or for example, the exemplary vaccinia cell fusion system described herein. If a compound blocks env-mediated membrane fusion (I) involved in HIV entry into a human CD4-positive target cell or (ii) between an HIV-infected cell and an uninfected human CD4-positive target cell, the compounds are suitable according to the invention. The preferred peptide fragments of STRL33 according to the invention include those which correspond to the regions of STRL33 that are exposed on the cell surface or that can be exposed following interaction with other molecules.

### STRL33-BINDING AND BLOCKING AGENTS

In yet another embodiment, the present invention relates to substantially purified STRL33-binding and/or blocking agents that block membrane fusion between HIV and a target cell. Such agents could represent research and diagnostic tools in the study of HIV infection and the development of more effective anti-HIV therapeutics. In addition, pharmaceutical compositions comprising isolated and purified STRL33-binding agents may represent effective anti-HIV therapeutics. The phrase “STRL33-binding agent” denotes the natural ligand of STRL33, a synthetic ligand of STRL33, or appropriate fragments of the natural or synthetic ligands which either bind to STRL33 or block STRL33 in HIV-env mediated membrane fusion. The term includes both biologic agents and chemical compounds. The determination and isolation of ligand/compositions is well described in the art. See, e.g., Lemer, Trends NeuroSci. 17:142-146 (1994). which is hereby incorporated in its entirety by reference.

Various chemokines may function as a biologic agent as a ligand for STRL33. Derivatives, analogs, mutants and STRL33 binding fragments of STRL33 ligand are useful for blocking env-mediated membrane fusion.

An STRL33-binding agent that blocks env-mediated membrane fusion (I) involved in HIV entry into a human CD4-positive target cell or (ii) between an HIV-infected cell and an uninfected human CD4-positive target cell, is suitable according to the invention.

### SCREEN FOR STRL33 BINDING AND BLOCKING COMPOSITIONS

In another embodiment, the invention provides a method for identifying a composition which binds to STRL33 or blocks HIV env-mediated membrane fusion. The method includes incubating components comprising the composition and STRL33 under conditions sufficient to allow the components to interact and measuring the binding of the composition to STRL33. Compositions that bind to STRL33 include peptides, peptidomimetics, polypeptides, chemical compounds and biologic agents as described above.

Incubating includes conditions which allow contact between the test composition and STRL33. Binding can be measured indirectly by biochemical alterations in the cell (e.g., calcium flux). Contacting includes in solution and in solid phase. The test ligand(s)/composition may optionally be a combinatorial library for screening a plurality of compositions. Compositions identified in the method of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology, 3:1008-1012, 1985), allele-specific oligonucleotide (ASO) probe analysis (Conner, et al., Proc. Natl. Acad. Sci. USA, 80:278, 1983), oligonucleotide ligation assays (OLAs) (Landegren, et al, Science, 241:1077, 1988), and the like. Molecular techniques for DNA analysis have been reviewed (Landegren, et al., Science, 242:229-237, 1988).

Any of a variety of procedures may be used to clone the genes of the present invention when the test composition is in a combinatorial library or is expressed as a gene product (as opposed to a chemical composition). One such method entails analyzing a shuttle vector library of DNA inserts (derived from a cell which expresses the composition) for the presence of an insert which contains the composition gene. Such an analysis may be conducted by transfecting cells with the vector and then assaying for expression of the composition binding activity. The preferred method for cloning these genes entails determining the amino acid sequence of the composition protein. Usually this task will be accomplished by purifying the desired composition protein and analyzing it with automated sequencers. Alternatively, each protein may be fragmented as with cyanogen bromide, or with proteases such as papain, chymotrypsin or trypsin (Oike, Y., et al., J. Biol. Chem., 257:9751-9758 (1982); Liu, C., et al., Int. J. Pept. Protein Res., 21:209-215 (1983)). Although it is possible to determine the entire amino acid sequence of these proteins, it is preferable to determine the sequence of peptide fragments of these molecules.

To determine if a composition can functionally complex with the receptor protein, induction of the exogenous gene is monitored by monitoring changes in the protein levels of the protein encoded for by the exogenous gene, for example. When a composition(s) is found that can induce transcription of the exogenous gene, it is concluded that this composition(s) can bind to the receptor protein coded for by the nucleic acid encoding the initial sample test composition(s).

Expression of the exogenous gene can be monitored by a functional assay or assay for a protein product, for example. The exogenous gene is therefore a gene which will provide an assayable/measurable expression product in order to allow detection of expression of the exogenous gene. Such exogenous genes include, but are not limited to, reporter genes such as chloramphenicol acetyltransferase gene, an alkaline phosphatase gene, beta-galactosidase,a luciferase gene, a green fluorescent protein gene, guanine xanthine phosphoribosyltransferase, alkaline phosphatase, and antibiotic resistance genes (e.g., neomycin phosphotransferase).

Expression of the exogenous gene is indicative of composition-receptor binding, thus, the binding or blocking composition can be identified and isolated. The compositions of the present invention can be extracted and purified from the culture media or a cell by using known protein purification techniques commonly employed, such as extraction, precipitation, ion exchange chromatography, affinity chromatography, gel filtration and the like. Compositions can be isolated by affinity chromatography using the modified receptor protein extracellular domain bound to a column matrix or by heparin chromatography.

Also included in the screening method of the invention is combinatorial chemistry methods for identifying chemical compounds that bind to STRL33. Ligands/compositions that bind to STRL33 can be assayed in standard cell:cell fusion assays, such as the vaccinia assay described herein to determine whether the composition inhibits or blocks env-mediated membrane fusion (i) involved in HIV entry into a human CD4-positive target cell or (ii) between an HIV-infected cell and an uninfected human CD4-positive target cell.

### MODULATION OF A T CELL RESPONSE

The invention also includes a method for modulating a T cell immune response utilizing STRL33 agonists or antagonists. The method includes treatment of conditions in which immune reactions are deleterious and suppression of such responses or immune reactions is desirable and conditions in which immune reactions are important and stimulation of such responses is desirable. As used herein, the term “modulating” means stimulating or inhibiting the response, depending on the situation. For example, it is envisioned that STRL33 agonists may be useful in recruiting and/or activating T cells which would enhance the immune response to a vaccine, stimulate a response for tumor rejection, or alter the response in a qualitative manner. Similarly, STRL33 antagonists may inhibit or depress an immune or inflammatory response where desirable, such as in graft rejection responses after organ and tissue transplantations, or autoimmune disease. Some of the commonly performed transplantation surgery today includes organs and tissues such as kidneys, hearts, livers, skin, pancreatic islets and bone marrow. However, in situations where the donors and recipients are not genetically identical, graft rejections can still occur. Autoimmune disorders refer to a group of diseases that are caused by reactions of the immune system to self antigens leading to tissue destruction. These responses may be mediated by antibodies, auto-reactive T cells or both. Some important autoimmune diseases include diabetes, autoimmnune thyroiditis, multiple sclerosis, rheumatoid arthritis, systemic lupus erythematosis, and myasthenia gravis.

### PHARMACEUTICAL COMPOSITIONS

The invention also contemplates various pharmaceutical compositions that block membrane fusion between HIV and a target cell. The pharmaceutical compositions according to the invention are prepared by bringing an antibody against STRL33, an isolated and purified peptide fragment of STRL33, or an isolated and purified STRL33-binding biologic agent according to the present invention into a form suitable for administration (e.g., a pharmaceutically acceptable carrier) to a subject using carriers, excipients and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol and polyhydric alcohols. Intravenous vehicles include fluid and nutrient replenishers. Preservatives include antimicrobial, anti-oxidants, chelating agents and inert gases. Other pharmaceutically acceptable carriers include aqueous solutions, non-toxic excipients, including salts, preservatives, buffers and the like, as described, for instance, in Remington's Pharmaceutical Sciences, 15th ed. Easton: Mack Publishing Co., 1405-1412, 1461-1487 (1975) and The National Formulary XIV., 14th ed. Washington: American Pharmaceutical Association (1975), the contents of which are hereby incorporated by reference. The pH and exact concentration of the various components of the pharmaceutical composition are adjusted according to routine skills in the art. See Goodman and Gilman's The Pharmacological Basis for Therapeutics (7th ed.).

In another embodiment, the invention relates to a method of blocking the membrane fusion between HIV and a target cell. This method involves administering to a subject a therapeutically effective dose of a pharmaceutical composition containing the compounds of the present invention and a pharmaceutically acceptable carrier. “Administering” the pharmaceutical composition of the present invention may be accomplished by any means known to the skilled artisan. By “subject” is meant any mammal, preferably a human.

The pharmaceutical compositions are preferably prepared and administered in dose units. Solid dose units are tablets, capsules and suppositories. For treatment of a patient, depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administration of subdivided doses at specific intervals.

The dosage should not be so large as to cause adverse side effects, such as unwanted cross-reactions, anaphylactic reactions and the like. Generally, the dosage will vary with the age, condition, sex, and extent of the disease in the patient and can be determined by one skilled in the art. The dosage can be adjusted by the individual physician in the event of any contraindications and can be readily ascertained without resort to undue experimentation. In any event, the effectiveness of treatment can be determined by monitoring the level of CD4+ T-cells in a patient. An increase or stabilization in the relative number of CD4+ cells should correlate with recovery of the patient's immune system.

The pharmaceutical compositions according to the invention are in general administered topically, intravenously, orally or parenterally or as implants, but even rectal use is possible in principle. Suitable solid or liquid pharmaceutical preparation forms are, for example, granules, powders, tablets, coated tablets, (micro)capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science, 249: 1527-1533 (1990), which is incorporated herein by reference.

The pharmaceutical compositions according to the invention may be administered locally or systemically. By “therapeutically effective dose” is meant the quantity of a compound according to the invention necessary to prevent, to cure or at least partially arrest the symptoms of the disease and its complications. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al. (eds.) (1990) GOODMAN AND GILMAN'S: THE PHARMACOLOGICAL BASES OF TIHERAPEUTICS, 8th ed., Pergamon Press; and REMINGTON'S PHARMACEUTICAL SCIENCES, 17th ed. (1990), Mack Publishing Co., Easton, Pa., each of which is herein incorporated by reference. Effectiveness of the dosage can be monitored by CD4+ count as described above in this section.

The pharmaceutical compositions of the invention, including antibodies, peptides, peptidomimetics, chemical compositions, etc., are all useful for treating subjects either having or at risk of having an HIV related disorder. AIDS and ARC are preferred examples of such disorders. HIV-associated disorders have been recognized primarily in “at risk” groups, including homosexually active males, intravenous drug users, recipients of blood or blood products, and certain populations from Central Africa and the Caribbean. The syndrome has also been recognized in heterosexual partners of individuals in all “at risk” groups and in infants of affected mothers.

The immunotherapeutic method of the invention includes a prophylactic method directed to those hosts at risk for the HIV infection. For example, the method is useful for humans at risk for HIV infection. A “prophylactically effective” amount of antibody or peptide, for example, refers to that amount which is capable of blocking env-mediated membrane fusion in HIV entry into a human CD4-positive target cell or between an HIV-infected cell and an uninfected human CD4-positive target cell.

Transmission of HIV occurs by at least three known routes: sexual contact, blood (or blood product) transfusion and via the placenta. Infection via blood includes transmission among intravenous drug users. Since contact with HIV does not necessarily result in symptomatic infection, as determined by seroconversion, all humans may be potentially at risk and, therefore, should be considered for prophylactic treatment by the therapeutic method of the invention.

The compositions described herein and useful in the method of the invention can be administered to a patient prior to infection with HIV (i.e., prophylactically) or at any of the stages described below, after initial infection. The HIV infection may run any of the following courses: 1) approximately 15% of infected individuals have an acute illness, characterized by fever, rash, and enlarged lymph nodes and meningitis within six weeks of contact with HIV. Following this acute infection, these individuals become asymptomatic. 2) The remaining individuals with HIV infection are not symptomatic for years. 3) Some individuals develop persistent generalized lymphadenopathy (PGL), characterized by swollen lymph nodes in the neck, groin and axilla. Five to ten percent of individuals with PGL revert to an asymptomatic state. 4) Any of these individuals may develop AIDS-related complex (ARC); patients with ARC do not revert to an asymptomatic state. 5) Individuals with ARC and PGL, as well as asymptomatic individuals, eventually (months to years later) develop AIDS which inexorably leads to death.

### GENE THERAPY

In yet another embodiment, the invention provides a method of treating a subject having or at risk of having an HIV-related disorder associated with expression of STRL33 comprising administering to an HIV infected or susceptible cell of the subject, a reagent that suppresses STRL33. Therapeutic methods of the invention using an anti-STRL33 antibody have been described above. The invention also includes methods of gene therapy wherein an antisense nucleic acid that hybridizes to a STRL33 nucleic acid is administered to a subject. The reagent is introduced into the cell using a carrier, such as a vector. Administration of the reagent can be in vivo or ex vivo.

This approach employs, for example, antisense nucleic acids (i.e., nucleic acids that are complementary to, or capable of hybridizing with, a target nucleic acid, e.g., a nucleic acid encoding a STRL33 polypeptide), ribozymes, or triplex agents. The antisense and triplex approaches function by masking the nucleic acid, while the ribozyme strategy functions by cleaving the nucleic acid. In addition, antibodies that bind to STRL33 polypeptides can be used in methods to block the entry of HIV into a cell or block cell fusion between HIV infected and uninfected cells.

The use of antisense methods to inhibit the in vitro translation of genes is well known in the art (see, e.g., Marcus-Sakura, Anal. Biochem., 172:289, 1988). Antisense nucleic acids are nucleic acid molecules (e.g., molecules containing DNA nucleotides, RNA nucleotides, or modifications (e.g., modification that increase the stability of the molecule, such as 2′-O-alkyl (e.g., methyl) substituted nucleotides) or combinations thereof) that are complementary to, or that hybridize to, at least a portion of a specific nucleic acid molecule, such as an RNA molecule (e.g., an mRNA molecule) (see, e.g., Weintraub, Scientific American, 262:40, 1990). The antisense nucleic acids hybridize to corresponding nucleic acids, such as mRNAs, to form a double-stranded molecule, which interferes with translation of the mRNA, as the cell will not translate an double-stranded mRNA. Antisense nucleic acids used in the invention are typically at least 10-12 nucleotides in length, for example, at least 15, 20, 25, 50, 75, or 100 nucleotides in length. The antisense nucleic acid can also be as long as the target nucleic acid with which it is intended that it form an inhibitory duplex. As is described further below, the antisense nucleic acids can be introduced into cells as antisense oligonucleotides, or can be produced in a cell in which a nucleic acid encoding the antisense nucleic acid has been introduced by, for example, using gene therapy methods.

In addition to blocking mRNA translation, oligonucleotides, such as antisense oligonucleotides, can be used in methods to stall transcription, such as the triplex method. In this method, an oligonucleotide winds around double-helical DNA in a sequence-specific manner, forming a three-stranded helix, which blocks transcription from the targeted gene. These triplex compounds can be designed to recognize a unique site on a chosen gene (Maher, et al., Antisense Res. and Dev., 1(3):227, 1991; Helene, Anticancer Drug Design, 6(6):569, 1991). Specifically targeted ribozymes can also be used in therapeutic methods directed at decreasing STRL33 expression.

Introduction of STRL33 antisense nucleic acids into cells affected by a proliferative disorder, for the purpose of gene therapy, can be achieved using a recombinant expression vector, such as a chimeric virus or a colloidal dispersion system, such as a targeted liposome. Those of skill in this art know or can easily ascertain the appropriate route and means for introduction of sense or antisense STRL33 nucleic acids, without resort to undue experimentation.

### HOMOZYGOUS AND HETEROZYGOUS MUTATIONS IN STRL33

It is known that in some cases, a homozygous or heterozygous mutation in a polypeptide or a regulatory region of a gene confers a molecular basis for a difference in function. Bertina, et al. and Greengard, et al. (Bertina, et al., Nature, 369:64, 1994; Greengard, et al., Lancet, 343:1361, 1994), first identified the molecular basis for the FV abnormality. The phenotype of APC resistance was shown to be associated with heterozygosity or homozygosity for a single point mutation in the FV gene that resulted in the substitution of arginine at amino acid residue 506 with glutamine (FV R506Q).

This R506Q mutation prevents APC from cleaving a peptide bond at Arg-506 in FV that is required to inactivate factor Va (Bertina, supra; Sun, et al., Blood, 83:3120, 1994).

Similarly, the present invention envisions diagnostic and prognostic, and in addition, therapeutic approaches to treatment of HIV-associated syndromes based on homozygosity or heterozygosity of STRL33 mutants. For example, while not wanting to be bound by a particular theory, it is believed that a subject having a homozygous mutant of STRL33 may be HIV resistant or exhibit a slower rate of disease progression. Along the same lines, a subject having a heterozygous mutation in STRL33 may exhibit a slower rate of disease progression than a patient having a wild type STRL33. Mutations included in the STRL33 coding region may also result in inactivating mutations. In addition, a mutation in the regulatory region of STRL33 gene may prevent or inhibit expression of STRL33, thereby providing resistance to some degree from HIV infection.

As described above, polymorphisms were identified in the ORF of STRL33. Specifically, a clone was identified as having second position change in the 25th codon (GAC→GCC) results in a substitution of aspartic acid with alanine. A silent change was also identified in codon 103.

Once an individual having a homozygous or heterozygous mutant in STRL33 is identified, it is envisioned that cells from that individual, once matched for histocompatibility, can be transplanted to an HIV positive individual, or to an “at risk” individual.

Without further elaboration, it is believed that one skilled in the art can, using the preceding description, utilize the present invention to its fullest extent. The following examples are illustrative only, and not limiting of the remainder of the disclosure in any way whatsoever.

### EXAMPLES

MATERIALS AND METHODS

Cell Culture. Jurkat, SUP-T1, U937, human embryonic kidney (HEK) 293, HeLa and NIH 3T3 cells were obtained from ATCC. CEM clone 12D7 was obtained from Dr. Keith Peden (CBER, FDA). Tumor infiltrating lymphocytes (TIL) R4, R8, F9 and B10, prepared from human melanomas, were obtained from Dr. John R. Yannelli, National Cancer Institute. EBV414 is an EBV-transformed B lymphoblastoid cell line obtained from Dr. Robert Siliciano, Johns Hopkins University. Jurkat, SUP-T1, CEM, U937 and EBV414 cells were grown in RPMI 1640 with 10% FBS. 293 cells were grown in MEM plus 10% horse serum. HeLa and NIH 3T3 cells were grown in DMEM with 10% FBS. TIL were grown in either RPMI 1640 with 10% FBS or in AIM-V (Life Technologies), in each case supplemented with 500 U/ml IL-2 and the cells were stimulated periodically with 250 ng/ml PHA plus irradiated allogeneic PBMC. Granulocytes were prepared as described and elutriated PBL and monocytes were prepared from normal donors by the Department of Transfusion Medicine, NIH.

Cloning of STRL33 cDNAs. Total RNA was prepared from the F9 TIL using TRIzol reagent (Life Technologies), poly(A)+ RNA was selected using oligo(dT) cellulose (Collaborative Biomedical Products), and first strand cDNA was synthesized using oligo(dT) primers and the SuperScript Preamplification System (Life Technologies) according to suppliers' protocols. For amplification, primer pools were designed based on transmembrane domain (TMD) II and TMD VII amino acid sequences from the human sequences for IL8RA, IL8RB, CCR1 and CCR2 and the murine homologues of IL8R and CCR1 and were 5′GA(T/C)(C/T)TI(C/T/G)TITT(T/C)(G/T/C)(C/T) I (T/C/A)TIACI(T/C)TICC, and 5′CCIA(T/C)(A/G)AAI (G/A)(C/T)(A/G) TAIA(T/A/G)IA(G/A/T/C)IGG(A/G)TT, respectively. Amplifications were done with cDNA synthesized from 0.015 mg of Poly(A)+ RNA, with 1.5 mM of each primer pool in a 20 ml reaction volume with Taq polymerase and reagents from Perkin Elmer according to the supplier's protocol. PCR was done using 30 cycles of denaturation at 94° C. for 0.5 min, annealing at 45° C. for 2 min and chain extension at 72° C. for 1.5 min.

One μl from the first PCR was used in a second PCR done identically to the first and the products of the second reaction were separated on a 1.5% agarose gel from which fragrnents of the approximate predicted size of 670 bp were purified and inserted by blunt end ligation into the vector pNOTA/T7 (5 Prime→3 Prime Inc.). Eighty eight ampicillin resistant bacterial transformants were picked and, to eliminate known sequences, hybridizations were done with radiolabelled oligonucleotide probes for receptors CCR1, CCR2, CCR3, fusin/CXCR4, BLR1, EBI1 and STRL22. Among the inserts in the non-hybridizing colonies was a novel sequence designated STRL33.

Using poly(A)+ RNA from F9 TIL a Lambda ZAP Express (Stratagene) cDNA library was prepared according to the supplier's protocol. 1.4×10⁶ recombinant phage from the non-amplified library were screened using a radiolabelled STRL33 probe. Ten positive phage were plaque-purified and the pBK-CMV (Stratagene) plasmids containing STRL33 inserts were recovered by in vivo excision according to the supplier's protocol. Manual and/or automated dideoxy sequencing was done for the entire cDNA clone STRL33.1, the 5′ non-translated region of clone STRL33.2, the 5′ non-translated region and open reading frame (ORF) of clone STRL33.3, and portions of other cDNA clones, some of which were obtained using RT-PCR.

Northern Blot Analysis. Total RNA was prepared as above. DNAs used for probes were: IL8RA, IL8RB, CCR3, EBI1 and BLR1 genomic fragments and CCR2B cDNA obtained from Dr. Philip Murphy, National Institute of Allergy and Infectious Diseases; STRL33, CCR1, CCR4, CCR5, CXCR4 and CMKBRL1 cDNAs that we isolated either from our lambda library or by RT-PCR from TIL mRNA; and an STRL22 genomic fragment isolated as described . Hybridizations to leukocyte RNA were performed as described with washes in 0.1×SSC, 0.1% SDS at 50° C. Hybridizations with an oligonucleotide probe to 18S rRNA were as described. The blot of poly(A)+ RNA from human tissues was obtained from Clontech (Palo Alto, Calif.). Hybridizations were done according to the supplier's protocol with washes as described above. Autoradiography/fluorography was done using an intensifying screen.

Production and analysis of STRL33-transfected cell lines. An EcoR I-Ear I fragment containing the complete STRL33 ORF was isolated from the pBK-CMV/STRL33.1 plasmid and inserted into pCEP4 (Invitrogen) and pCIneo (Promega Corp.). The pCEP4/STRL33 DNA, pCEP4 without a cDNA insert, and the pCIneo/STRL33 DNA were transfected into HEK 293 cells by calcium phosphate precipitation and into Jurkat cells by electroporation. Selection was in 200 μ/ml hygromycin B (Sigma) and 1 mg/ml G418 (Life Technologies) for pCEP4 transfected cells and pCIneo transfected cells respectively. Individual colonies of resistant 293 cells were cloned and expanded and Jurkat lines were derived by limiting dilution after the electroporation. Lines expressing the highest levels of STRL33 mRNA were used to test responses to chemokines using the fluorometric calcium flux assay as described . Recombinant HuMig was obtained by infecting High Five cells of Trichoplusia ni (Invitrogen), as will be described elsewhere, and was purified by column chromatography as described . IP-10, MCP-1, MCP-2, MCP-3, RANTES, MIP-1a, MIP-1b, Platelet factor 4, IL-8, and lymphotactin, were purchased from Pepro Tech. MCP-4 was a gift from Dr. Andrew Luster, Harvard University. I309 and SDF-1 were gifts from R & D Systems Co.

Assays for activity of STRL33 as a fusion cofactor. Assays were done using an E. coli lacZ reporter gene assay for fusion between two cell populations, one expressing an HIV-1 Env and the other expressing CD4 . Using DOTAP lipofectin (Boehringer Mannheim), NIH 3T3 cells were transfected with 10 μg of DNA, either pCIneo (Promega Corp.) containing the complete STRL33 ORF inserted downstream of the T7 promoter, or pCIneo lacking STRL33, or pCDNA3-fusin/CXCR4 or pGA9-CKR5 (encoding CCR5)(11). After 4-5 hours, the transfected cells were infected at 10 pfu/cell with recombinant vaccinia viruses vCB-3 encoding human CD4 and vTF7-3 encoding T7 RNA polymerase. A separate population of HeLa cells was co-infected with vaccinia virus vCB-21R-Lac Z containing Lac Z encoding β-galactosidase (β-Gal), under control of a T7 promoter and one of the following Env-encoding vaccinia viruses: vCB-41 encoding the LAV Env, vCB-39 encoding the ADA Env, vCB-28 encoding the JR-FL Env, vCB-32 encoding the SF-162 Env, vCB-43 encoding the Ba-L Env, vSC60 encoding the IIIB Env (S. Chakrabarti and B. Moss, personal communication), and vCB- 16 encoding the non-fusogenic Unc Env . Infected cells were incubated overnight at 31° C. Duplicate samples of 10⁵ transfected and infected NIH 3 T3 target cells and 10⁵ infected Env-expressing cells were mixed; after 2.5 h cells were lysed and b-Gal activity was measured as described.

Similar assays were performed to detect Env-mediated fusion with Jurkat cell lines that had been derived, as described above, following transfection with STRL33 sequences. Jurkat cell line JC3.9 transfected with pCEP4 containing the STRL33.1 cDNA and Jurkat cell line JC0.1 transfected with pCEP4 lacking STRL33 were infected with vaccinia virus vTF7-3 (encoding T7 RNA polymerase) and VCB-3 (encoding human CD4). Following overnight incubation, the infected cells were mixed with Env-expressing HeLa cells for analyzing fusion as described above.

References

1. Savarese, T. M., and C. M. Fraser. 1992. In vitro mutagenesis and the search for structure-function relationships among G protein-coupled receptors. Bioch. J. 283:1-19.

2. Murphy, P.1994. The molecular biology of leukocyte chemoattractant receptors. Ann. Rev. Immunol. 12:593-633.

3. Schall, T. J., and K. Bacon. 1994. Chemokine, leukocyte trafficking and inflammation. Curr. Opin. Immunol. 6:865-873.

4. Raport, C. J., V. L. Schweickart, D. Chantry, R. L. J. Eddy, T. Shows, R. Godiska, and P. W. Gray. 1996. New members of the chemokine receptor gene family. J. Leuk. Biol. 59:18-23.

5. Miedema, F., C. Meyaard, M. Koot, M. R. Klein, M. T. Roos, M. Groenink, R. A. Fouchier, and A. Van't Wout. 1994. Changing virus-host interactions in the course of HIV-1 infection. Immunol. Rev. 140:35-72.

6. Goudsmit, J. 1995. The role of viral diversity in HIV pathogenesis. J. Acq. Immun. Defic. Synd. Hum. R. 10:S15-S19.

7. Cocchi, F., A. L. DeVico, A. Garzino-Demo, S. K. Arya, R. C. Gallo, and P. Lusso. 1995. Identification of RANTES, MIP-1a, and MIP-1b as the major HIV-suppressive factors produced by CD8+ T cells. Science 270:1811-1815.

8. Feng, Y., C. C. Broder, P. E. Kennedy, and E. A. Berger 1996. HIV-1 entry cofactor: functional cDNA cloning of a seven-transmembrane, G-protein-coupled receptor. Science 272:872-877.

9. Bleul, C. C., M. Farzan, H. Choe, C. Parolin, I. Clark-Lewis, J. Sodroski, and T. A. Springer. 1996. The lymphocyte chemoattractant SDF-1 is a ligand for LESTER/fusin and blocks HIV-1 entry. Nature 382:829-833.

10. Oberlin, E., A. Amara, F. Bachelerie, C. Bessia, J.-L. Virelizier, F. Arenzana-Seisdedos, O. Schwartz, J.-M. Heard, I. Clark-Lewis, D. F. Legler, M. Loetscher, M. Baggiolini, and B. Moser 1996. The CXC chemokine SDF-1 is the ligand for LESTR/fusin and prevents infection by T-cell-line-adapted HIV-1. Nature 382:833-835.

11. Alkhatib, G., C. Combadiere, C. C. Broder, Y. Feng, P. E. Kennedy, P. M. Murphy, and E. A. Berger. 1996. CC CKR5: A RANTES, MIP-1a, MIP-1b receptor as a fusion cofactor for macrophage-tropic HIV- 1. Science 272:1955-1958.

12. Deng, H., R. Liu, W. Ellmeier, S. Choe, D. Unutmaz, M. Burkhart, P. Di Marzio, S. Marmon, R. E. Sutton, C. M. Hill, C. B. Davis, S. C. Peiper, T. J. Schall, D. R. Littman, and N. R. Landau. 1996. Identification of a major co-receptor for primary isolates of HIV-1. Nature 381:661-666.

13. Choe, H., M. Farzan, Y. Sun, N. Sullivan, B. Rollins, P. D. Ponath, L. Wu, C. R. Mackay, G. LaRosa, W. Newman, N. Gerard, C. Gerard, and J. Sodroski. 1996. The b-chemokine receptors CCR3 and CCR5 facilitate infection by primary HIV-1 isolates. Cell 85:1135-1148.

14. Doranz, B. J., J. Rucker, Y. Yi, R. J. Smyth, M. Samson, S. C. Peiper, M. Parmentier, R. G. Collman, and R. W. Doms. 1996. A dual-tropic primary HIV-1 isolate that uses fusin and the b-chemokine receptors CKR-5, CKR-3, and CKR-2b as fusion cofactors. Cell 85:1149-1158.

15. Dragic, T., V. Litwin, G. P. Allaway, S. R. Martin, Y. Huang, K. A. Nagashima, C. Cayanan, P. J. Maddon, R. A. Koup, J. P. Moore, and W. A. Paxton. 1996. HIV-1 entry into CD4+ cells is mediated by the chemokine receptor CC-CKR-5. Nature 381:667-673.

16. Simmons, G., D. Wilkinson, J. D. Reeves, M. T. Dittmar, S. Beddews, J. Weber, G. Carnegie, U. Desselberger, P. W. Gray, R. A. Weiss, and P. R. Calpharn. 1996. Primary, syncytium-inducing human immunodeficiency virus type I isolates are dual-tropic and most can use either Lestr or CCR-5 as co-receptors for virus entry. J. Virol. 70:8355-8360.

17. Samson, M., F. Libert, B. J. Doranz, J. Rucker, C. Liesnard, C.-M. Farber, S. Saragosti, C. Lapoumeroulie, J. Cognaux, C. Forceille, G. Muyldermans, C. Verhofstede, G. Burtonboy, M. Georges, T. Imai, S. Rana, Y. Yi, R. J. Smyth, R. G. Collman, R. W. Doms, G. Vassart, and M. Parmentier. 1996. Resistance to HIV-1 infection in caucasian individuals bearing mutant alleles of the CCR-5 chemokine receptor gene. Nature 382:722-725.

18. Liu, R., W. A. Paxton, S. Choe, D. Ceradini, S. R. Martin, R. Horuts, M. E. MacDonald, H. Stahlmann, R. A. Koup, and N. R. Candau. 1996. Homozygous defect in HIV-1 coreceptor accounts for resistance of some multiply-exposed individuals to HIV-1 infection. Cell 86:367-377.

19. Dean, M., M. Carrington, C. Winkler, G. A. Huttley, M. W. Smith, R. Allikmets, J. J. Goedert, S. P. Buchbinder, E. Vittinghoff, E. Gomperts, S. Donfield, D. Vlahov, R. Kaslow, A. Saah, C. Rinaldo, R. Detels, M.A.C.S. Hemophilia Growth and Development Study, Multicenter Hemophilia Cohort Study, A. S. San Francisco City Cohort, and S. J. O'Brien. 1996. Genetic restriction of HIV-1 infection and progression to AIDS by a deletion allele of the CKR5 structural gene. Science 273:1856-1862.

20. Zimmerman, P. A., A. Buckler-White, G. Alkhatib, T. Spalding, J. Kubofcik, C. Combadiere, D. Weissman, O. Cohen, A. Rubbert, G. Lam, M. Vaccarezza, P. E. Kennedy, V. Kumraraswami, J. V. Gorgi, R. Detels, J. Hunter, M. Chopek, E. A. Berger, A. S. Fauci, T. B. Nutman, and P. M. Murphy. 1996. Inherited resistance to HIV-1 conferred by an inactivating mutation in CC chemokine receptor 5: studies in populations with contrasting clinical phenotypes, defined racial background and quantified risk. Molecular Medicine. In press.

21. Clark, R. A., and W. M. Nauseef. 1991. Isolation and finctional analysis of neutrophils. In Current protocols in Immunology. J. E. Coligan, A. M. Kruisbeek, D. H. Margulies, E. M. Shevach, and W. Strober. John Wiley and Sons, New York. 7.23.1-7.23.3.

22. Liao, F., H.-H. Lee, and J. M. Farber. 1996. Cloning of STRL22, a new human gene encoding a G protein-coupled receptor related to chemokine receptors and located on chromosome 6q27. Genomics. In press.

23. Vanguri, P., and J. Farber. 1990. Identification of CRG-2: An interferon-inducible mRNA predicted to encode a murine monokine. J. Biol. Chem. 265:15049-15057.

24. Amichay, D., R. T. Gazzinelli, G. Karupiah, T. R. Moench, A. Sher, and J. M. Farber. 1996. The gene for chemokines MuMig amd Crg-2 are induced in protozoan and viral infections in response to IFN-g with patterns of tissue expression that suggest nonredundant roles in vivo. J. Immunol. 157:4511-4520.

25. Kingston, R. E. 1996. Transfection of DNA into eukaryotic cells. In Current Protocol in Molecular Biology. R. M. Ausubel, R. Brent, R. E. Kingston, D. D. Moore, J. G. Seidman, J. A. Srnith, and K, Struhl John Wiely and Sons, New York. 9.11-9.19. 9.11-9.19.

26. Memon, S. A., D. Petrak, M. B. Moreno, and C. M. Zacharchuk. 1995. A simple assay for examining the effect of transiently expressed genes on programmed cell death. J. Immunol. Methods 180:15-24.

27. Liao, F., R. L. Rabin, J. R. Yannelli, L. G. Konianris, P. Vanguri, and J. M. Farber. 1995. Human Mig chemokine: biochemical and functional characterization. J. Exp. Med. 182:1301-1314.

15 28. Nussbaum, O., C. C. Broder, and E. A. Berger. 1994. Fusogenic mechanisms of enveloped-virus glycoproteins analyzed by a novel recombinant vaccinia virus-based assay quantitating cell fusion-dependent reporter gene activation. J. Virol. 68:5411-5422.

29. Broder, C. C., D. S. Dimitrov, R. Blumenthal, and E. A. Berger. 1993. The block to HIV-1 envelope glycoprotein-mediated membrane fusion in animal cells expressing human CD4 can be overcome by a human cell component(s). Virology 193:483-491.

30. Fuerst, T. R., E. G. Niles, F. W. Studier, and B. Moss. 1986. Eukaryotic transient-expression system based on recombinant vaccinia virus that synthesized bacteriophage T7 RNA polymerase. Proc. Natl. Acad. Sci. USA 83:8122-8126.

31. Alkhatib, G., C. C. Border, and E. A. Berger. 1996. Cell type-specific cofactors determine human immunodeficiency virus type I tropism for T-cell lines versus primarily macrophages. J. Virol. 70:5478-5494.

32. Broder, C. C., and E. A. Berger. 1995. Fusogenic selectivity of the envelope glycoprotein is a major determinant of human immunodeficiency virus type 1 tropism for CD4+ T-cell lines vs. primary macrophages. Proc. Natl. Acad. Sci. USA 92:9004-9008.

33. Kozak, M. 1987. An analysis of 5′-noncoding sequences from 699 vertebrate messenger RNAs. Nuc. Acid. Res. 15:8125-8148.

34. Altschul, S. F., W. Gish, W. Miller, E. W. Mayers, and D. J. Lipman. 1990. Basic local alignment search tool. J. Mol. Biol. 215:403-410.

35. Birkenbach, M., K. Josefsen, R. Yalamanchili, G. Lenoir, and E. Kieff. 1993. Epstein-Barr virus-induced genes: first lymphocyte-specific G protein-coupled peptide receptors. J. Virol. 67:2209-2220.

What is claimed is:

1. A recombinant cell line that expresses STRL33 polypeptide.

2. The cell line of claim 1, wherein the cell further expresses CD4 polypeptide.

3. A recombinant host cell stably transformed with a polynucleotide encoding STRL33 polypeptide, wherein the cell co-expresses STRL33 and CD4 polypeptide.

4. A recombinant host cell stably transformed with a polynucleotide encoding STRL33 polypeptide and a polynucleotide encoding CD4 polypeptide, wherein the cell co-expresses STRL33 and CD4 polypeptide.

5. The cell as in any of claims 1-4, wherein the cell is a human cell.

6. The cell as in any of claims 1-4, wherein the cell is a non-human cell.

7. An antibody which specifically binds to STRL33 polypeptide or fragments thereof.

8. The antibody of claim 7, wherein the antibody is a monoclonal antibody.

9. A substantially purified peptide fragment of STRL33, wherein the peptide inhibits cell membrane fusion between HIV and a target cell or between an HIV-infected cell and a CD4 positive uninfected cell.

10. A substantially purified STRL33-binding agent, wherein the biologic agent inhibits membrane fusion between HIV and a target cell or between an HIV-infected cell and a CD4 positive uninfected cell.

11. The agent of claim 10, wherein the agent is selected from a biologic agent and a chemical compound.

12. The agent of claim 10, wherein the biologic agent is a chemokine.

13. The agent of claim 12, wherein the agent is STRL33 ligand derivative, analog or binding fragment thereof.

14. A method of inhibiting membrane fusion between HIV and a target cell or between an HIV-infected cell and a CD4 positive uninfected cell comprising contacting the target or CD4 positive cell with a fusion-inhibiting effective amount of a STRL33 binding or blocking agent.

15. The method of claim 14, wherein the agent is STRL33 ligand or derivative, analog or binding fragment thereof.

16. The method of claim 14, wherein the agent is a anti-STRL33 antibody or epitope binding fragment thereof.

17. The method of claim 16, wherein the antibody is a monoclonal antibody or a polyclonal antibody.

18. The method of claim 14, wherein the contacting is by in vivo administration to a subject.

19. The method of claim 18, wherein the anti-STRL33 antibody is administered by intravenous, intra-muscular or subcutaneous injections.

20. The method of claim 19, wherein the anti-STRL33 antibody is administered within a dose range of 0.1 ug/kg to 100 mg/kg.

21. The method of claim 16, wherein the antibody is formulated in a pharmaceutically acceptable carrier.

22. A method for identifying a composition which binds to STRL33 polypeptide comprising:
a) incubating components comprising the composition and STRL33 polypeptide under conditions sufficient to allow the components to interact; and
b) measuring the binding or effect of binding of the composition to STRL33 polypeptide.

23. The method of claim 22, wherein the composition is a peptide.

24. The method of claim 22, wherein the composition is a peptidomimetic.

25. The method of claim 22, wherein the STRL33 polypeptide is expressed in a cell.

26. The method of claim 25, wherein the cell is the cell of claim 1.

27. A method for identifying a composition which blocks membrane fusion between HIV and a target cell or between an HIV-infected cell and a STRL33 positive uninfected cell comprising:
a) incubating components comprising the composition and a STRL33 positive cell under conditions sufficient to allow the components to interact;
b) contacting the components of step a) with HIV or an HIV-infected cell; and
c) measuring the ability of the composition to block membrane fusion between HIV and the STRL33 positive cell or between an HIV-infected cell and a STRL33 positive uninfected cell.

28. The method of claim 27, wherein the STRL33 positive cell is a CD4 positive cell.

29. The method of claim 27, wherein measuring the ability of the composition to block membrane fusion is by detection of a reporter means.

30. The method of claim 29, wherein the reporter means is selected from the group consisting of a radioisotope, a fluorescent compound, a bioluminescent compound, a chemiluminescent compound, a metal chelator, or an enzyme.

31. The method of claim 30, wherein the reporter means is a lacZ gene.

32. A transgenic non-human animal having a phenotype characterized by expression of STRL33 polypeptide and CD4 polypeptide otherwise not naturally occurring in the animal, the phenotype being conferred by a transgene contained in the somatic and germ cells of the animal, the transgene comprising a nucleic acid sequence which encodes STRL33 polypeptide and a nucleic acid sequence which encodes CD4 polypeptide.

33. The transgenic non-human animal of claim 32, wherein the animal is a mouse.

34. The transgenic non-human animal of claim 32, wherein the animal is a rabbit.

35. A transgenic non-human animal having a phenotype characterized by expression of STRL33 polypeptide otherwise not naturally occurring in the animal, the phenotype being conferred by a transgene contained in the somatic and germ cells of the animal, the transgene comprising a nucleic acid sequence which encodes STRL33 polypeptide.

36. A method for producing a transgenic non-human animal having a phenotype characterized by expression of STRL33 polypeptide and CD4 polypeptide otherwise not naturally occurring in the animal, the method comprising:
(a) introducing at least one transgene into a zygote of an animal, the transgene(s) comprising a DNA construct encoding STRL33,
(b) transplanting the zygote into a pseudopregnant animal,
(c) allowing the zygote to develop to term, and
(d) identifying at least one transgenic offspring containing the transgene.

37. The method of claim 36, further comprising a DNA construct encoding CD4.

38. The method of claim 36, wherein the introducing of the transgene into the embryo is by introducing an embryonic stem cell containing the transgene into the embryo.

39. The method of claim 36, wherein the introducing of the transgene into the embryo is by infecting the embryo with a retrovirus containing the transgene.

40. The method of claim 36, wherein the animal is selected from the group consisting of a mouse and a rabbit.

41. A transgenic non-human animal having a transgene disrupting or interfering with expression of STRL33 chromosomally integrated into the germ cells of the animal.

42. The transgenic animal of claim 41, wherein the animal is selected from the group consisting of a mouse and a rabbit.

43. The transgenic non-human animal of claim 41, wherein the transgene comprises STRL33 antisense polynucleotide.

44. A method of treating a subject having or at risk of having an HIV infection or disorder, comprising administering to the subject, a therapeutically effective amount of an anti-STRL33 antibody, wherein the antibody inhibits cell-cell fusion in cells infected with HIV.

45. The method of claim 44, wherein the antibody is a monoclonal antibody.

46. The method of claim 45, wherein the monoclonal antibody is a humanized monoclonal antibody.

47. The method of claim 44, wherein the monoclonal antibody is administered to a patient suffering from AIDS or ARC.

48. The method of claim 44, wherein the monoclonal antibody is administered within a dose range between about 0.1/kg to about 100 mg/kg.

49. The method of claim 44, wherein the monoclonal antibody is formulated in a pharmaceutically acceptable carrier.

50. A method of treating a subject having an HIV-related disorder associated with expression of STRL33 comprising administering to an HIV infected or susceptible cell of the subject, an agent that suppresses STRL33.

51. The method of claim 50, wherein the agent is an anti-STRL33 antibody.

52. The method of claim 50, wherein the agent is an antisense nucleic acid that hybridizes to a STRL33 nucleic acid.

53. The method of claim 50, wherein the agent is introduced into the cell using a carrier.

54. The method of claim 50, wherein the carrier is a vector.

55. The method of claim 50, wherein the administering is ex vivo.

56. The method of claim 50, wherein the administering is in vivo.

57. A method for detecting susceptibility of a first cell to HIV infection comprising:
incubating the first cell with a second cell which expresses HIV-env, under conditions to allow fusion of the two cells; and detecting fusion of the cells, wherein fusion is indicative of susceptibility to HIV infection.

58. The method of claim 57, wherein the first or second cell further comprises a reporter means for detection of cell fusion.

59. The method of claim 57, wherein the first cell is a T cell.

60. The method of claim 58, wherein the T-cell is a STRL33− and CD4+ cell.

61. The method of claim 58, wherein the T-cell is a STRL33+ and CD4− cell.

62. The method of claim 57, wherein the T-cell is a STRL33+ and CD4+ cell.

63. The method of claim 58, wherein the reporter means is selected from the group consisting of a radioisotope, a fluorescent compound, a bioluminescent compound, a chemiluminescent compound, a metal chelator, or an enzyme.

64. The method of claim 63, wherein the reporter means is a lacZ gene.

65. An isolated nucleic acid sequence comprising a polynucleotide sequence encoding a polypeptide having an amino acid sequence of FIG. 4.

66. The isolated nucleic acid sequence of claim 65, comprising a polynucleotide sequence encoding a polypeptide having an amino acid sequence of FIG. 4 and having at least one epitope for an antibody immunoreactive with STRL33.

67. An isolated nucleic acid sequence, wherein the nucleotide sequence is selected from the group consisting of:
a) FIG. 4, wherein the nucleotide T can also be the nucleotide U];
b) nucleic acid sequences encoding a polypeptide according to FIG. 4;
c) fragments of a) or b) that are at least 15 bases in length and which will selectively hybridize under stringent hybridization conditions to genomic DNA which encodes STRL33;
d) nucleotide sequences which encode polypeptides with conservative variations from the amino acid sequences of a), b) or c); and
e) functional fragments of a), b), c) or d) which retain the biological activity of STRL33.

68. A recombinant expression vector which contains the nucleic acid sequence of claim 65.

69. A host cell which contains the vector of claim 68.

70. Substantially pure STRL33 polypeptide.

71. An antibody which bind to the polypeptide of claim 70.

72. The antibody of claim 71, wherein the antibody is monoclonal.
